(19) United States
(12) Patent Application Publication
(10) Pub. No.: US 2013/0266937 A1
(43) Pub. Date: Oct. 10, 2013

(54) COMPOSITIONS AND METHODS FOR DIAGNOSING AND TREATING MACULAR DEGENERATION

(71) Applicants: Anand Swaroop, Ann Arbor, MI (US); Goncalo Abecasis, Ann Arbor, MI (US); Atsuhiro Kanda, Ann Arbor, MI (US); Mingyao Li, Philadelphia, PA (US)

(72) Inventors: Anand Swaroop, Ann Arbor, MI (US); Goncalo Abecasis, Ann Arbor, MI (US); Atsuhiro Kanda, Ann Arbor, MI (US); Mingyao Li, Philadelphia, PA (US)

(73) Assignee: THE REGENTS OF THE UNIVERSITY OF MICHIGAN, Ann Arbor, MI (US)

(21) Appl. No.: 13/785,942

(22) Filed: Mar. 5, 2013

Related U.S. Application Data
(62) Division of application No. 12/197,855, filed on Aug. 25, 2008.
(60) Provisional application No. 60/957,959, filed on Aug. 24, 2007.
(60) Provisional application No. 60/970,089, filed on Sep. 5, 2007.
(60) Provisional application No. 61/035,303, filed on Mar. 10, 2008.

(51) Int. Cl. C12Q 1/68

(52) U.S. Cl. CPC C12Q 1/6883
USPC 435/6.11

(57) ABSTRACT

The present invention relates generally to biomarkers for macular degeneration. In particular, the present invention provides a plurality of biomarkers (e.g., polymorphisms and/or haplotypes) for monitoring and diagnosing macular degeneration. The compositions and methods of the present invention find use in diagnostic, therapeutic, research, and drug screening applications.

### CROSS REFERENCE TO RELATED APPLICATIONS

The present application is a divisional of U.S. patent application Ser. No. 12/197,855, filed Aug. 25, 2008, which claims priority to U.S. Provisional Patent Application Ser. Nos. 60/957,959 filed Aug. 24, 2007, 60/970,089 filed Sep. 5, 2007 and 61/035,303 filed Mar. 10, 2008, each of which are herein incorporated by reference in its entirety.

### STATEMENT REGARDING FEDERALLY SPONSORED RESEARCH OR DEVELOPMENT

This invention was made with government support under Grant No EY016862 awarded by the National Institutes of Health. The Government has certain rights in the invention.

### FIELD OF THE INVENTION

The present invention relates generally to biomarkers for macular degeneration. In particular, the present invention provides a plurality of biomarkers (e.g., polymorphisms and/or haplotypes) for monitoring and diagnosing macular degeneration. The compositions and methods of the present invention find use in diagnostic, therapeutic, research, and drug screening applications.

### BACKGROUND OF THE INVENTION

Age-related macular degeneration (AMD; OMIM 603075) is a complex degenerative disorder that primarily affects the elderly. Disease susceptibility is influenced by multiple genetic^{1, 2, 3, 4, 5} and environmental factors^{6, 7, 8, 9}. Recently, targeted and genome-wide searches have identified alleles on chromosomes 1q and 10q that are strongly associated with disease susceptibility^{10, 11, 12, 13, 14}. In each case, the association appears robust and has been replicated in multiple samples. It has been documented that the Y402H-encoding variant of CFH is strongly associated with AMD susceptibility in a sample of affected individuals and controls. However, additional factors related to the susceptibility to AMD remain unknown.

### SUMMARY OF THE INVENTION

The present invention relates generally to biomarkers for macular degeneration. In particular, the present invention provides a plurality of biomarkers (e.g., polymorphisms and/or haplotypes) for monitoring and diagnosing macular degeneration. The compositions and methods of the present invention find use in diagnostic, therapeutic, research, and drug screening applications. The present invention further provides assay for identifying, characterizing, and testing therapeutic agents that find use in treating macular degeneration.

For example, in some embodiments, the present invention provides compositions (e.g., reagents, kits, reaction mixtures, etc. useful for, necessary for, or sufficient for carrying out the methods described herein) and methods for characterizing a subject's risk for developing age-related macular degeneration (AMD). In some embodiments the methods comprise detecting the presence of or the absence of one or more (e.g., two or more, three or more, four or more, five or more, etc.) polymorphisms selected from the group rs2274700, rs1410996, rs7535263, rs10801559, rs3766405, rs10754199, rs1329428, rs10922104, rs1887973, rs10922105, rs4658046, rs10465586, rs3753395, rs402056, rs7529589, rs7514261, rs10922102, rs10922103, rs800290, rs1061147, rs1061170, rs1048663, rs412852, rs11582939, and rs1280514. In some embodiments, the polymorphism(s) displays stronger association with disease susceptibility than the Y402H variant. In some embodiments, the polymorphism(s) does not change CFH protein. Where two or more such markers are used, any one of them may be used in combination with any other. For example, rs3766405 may be used alone or in combination with any one or more of the other markers. Panels, containing two or more markers may contain one or more of the above markers in combination with one or more other markers of macular degeneration or other diseases or conditions of interest to a physician or patient. In some embodiments, the method detects the presence of or the absence of one or more polymorphisms and/or variants found in LOC387715/ARMS2 (e.g., rs10490924 and/or polymorphisms in linkage disequilibrium therewith). ARMS2 markers may be detected alone or in combination with any of the above described markers.

The present invention also provides compositions and methods for characterizing agents for treating macular degeneration. Any one or more of the markers may be used in such methods. For example, in some embodiments, the method comprises exposing an organism, tissue, or cell to an agent and assessing a change in an ARMS2 (or other marker) biological activity. In some embodiments, the organism, tissue, or cell comprises a heterologous ARMS2 gene (or other marker). In some embodiments, the organism, tissue, or cell does not normally comprise the marker gene (e.g., ARMS2 is expressed in a non-primate such as a rodent). In some embodiments, the change in biological activity is a change in marker expression (mRNA or protein). In some embodiments, the biological activity is a change in cell function (e.g., mitrochondrial function). In some embodiments, the biological activity is a change in organism function (e.g., tissue health, signs or symptoms of disease).

### DESCRIPTION OF THE DRAWINGS

FIG. 1 shows P values for single-SNP association, when comparing unrelated affected individuals (cases) and controls. The dotted horizontal line is −log₁₀(P) of the original Y402H variant. Strongly associated SNPs fall into one of two LD groups (SNPs in one of these groups are represented as small squares; SNPs in the other group are represented as small triangles; SNPs outside either group represented as small filled circles). SNPs selected from the stepwise haplotype association analysis are circled in red. Linkage disequilibrium across the CFH region²⁹ is shown below, plotted as pairwise r² values.

FIG. 2 shows effects of rs1061170 (Y402H) and 20 SNPs showing even more significant association with AMD and SNPs selected in the stepwise haplotype analysis. The rs number for each SNP (as provided by the NCBI dbSNP database, available at web site ncbi.nlm.nih.gov/projects/SNP/, hereby incorporated by reference) is followed by its risk allele (defined as the allele with higher frequency in affected individuals than in controls) and position in the May 2004 genome assembly. Association analyses are summarized for a sample of unrelated individuals and, in addition, for the full sample including multiple affected relative pairs. N is the number of genotypes available among unrelated individuals; LRT is the standard likelihood ratio test statistic used to compare allele frequencies in cases and controls. Affect., affected individuals; ctrl., controls; When analyzing the full sample, a χ² statistic corresponding to a parametric model of association was calculated using the LAMP16,17 program. The frequency of the risk allele in the population, penetrances for each genotype, and λ_{sib} (ref. 18) for each SNP as estimated by LAMP are tabulated. The associated markers fall in two LD groups. Markers in each group have r²>0.80 with each other and markers in different groups have r² of ˜0.40 with each other. The table includes association results for the 20 SNPs that show stronger association than rs1061170 (the Y402H variant) and four additional SNPs that show weaker marginal association but that were included in the haplotype model.

FIG. 3 shows results of stepwise haplotype association analysis. Empirical P value was adjusted for multiple testing and was assessed using 10,000 permutations. A permutated sample was obtained by permuting disease affection status among affected individuals and controls while preserving evidence for association among SNPs selected in the previous step. Specifically, at each step, individuals were grouped according to genotype patterns at previously selected SNPs, and then the disease affection status was permuted within each group of individuals with the same genotype pattern. Haplotype association was evaluated using a likelihood ratio test to compare haplotype frequencies between cases and controls. The likelihood ratio statistic was calculated with FUGUE-CC28. DLRT, difference in the likelihood ratio statistic between the current step and the previous step.

FIG. 4 shows association analysis of selected 5-SNP haplotypes. Haplotype frequencies estimated using PHASE30. All haplotypes with frequency >1% in the combined case and control sample are shown. Haplotypes with a frequency <0.05 were pooled before haplotype trend regression. Putative risk haplotypes are marked in bold. A,2 between Y402H and each of the five haplotype groups (four common haplotypes and one pool of rare haplotypes) is ˜0.78, 0.41, 0.03, 0.08 and 0.00. D′ is ˜0.96, 1.00, 1.00, 1.00 and 0.02. When cases and controls were examined separately, the frequency of allele C at Y402H was 0.96 in affected individuals and 0.89 in controls (for carriers of haplotype 1), and 0.40 in affected individuals and 0.31 in controls (for carriers of one of the rare haplotypes).

FIG. 5 shows estimated probability of disease for each possible haplo-genotype combination. Probabilities estimated using maximum likelihood and assuming a multiplicative model for disease risk. s.d. for each estimate (in parenthesis) estimated using the jackknife procedure. Population prevalence was fixed at 20%. h1-h8 represent the eight haplotypes listed in FIG. 4.

FIG. 6 shows analysis of Y402H and of SNPs selected in a stepwise search using the haplotype method of Valdes and Thomson (1997). The method of Valdes and Thomson (1997) compares haplotypes that carry a putative disease allele in cases and controls. If there are no other disease alleles in the region (or else, if they are all in complete LD with the original variant) there should be no systematic differences between the case and control haplotypes. As shown in the top panels of the figure, both for the Y402H variant and for rs2774700, haplotypes appear to be quite different in cases and controls (the large dot is the original statistic and the small dots are statistics from 1000 permuted datasets). The method can also determine whether haplotypes defined using a set of SNPs perfectly distinguish all the disease alleles in a region. If they do, there should be no systematic differences between cases and controls at haplotypes classified using these markers. The middle two panels show that when case and control haplotypes are classified using the best two or three SNPs only, there is still evidence for additional disease associated alleles. The bottom two panels show the evidence is much weaker once 4 or 5 SNPs are included in the haplotype model, since the observed data point is no longer an extreme outlier but instead falls at the edge of the cloud of permuted points (See Table 2 and Equation 9 of Valdes and Thomson Am J Hum Genet 60, 703-16 (1997)).

FIG. 7 shows sensitivity of LAMP results to estimates of disease prevalence. FIG. 7 summarizes likelihood ratio test (LRT) statistics obtained from LAMP for association analyses assuming different estimates of the disease prevalence (K). All analyses give very similar results.

FIG. 8 shows association test results for all SNPs.

FIG. 9 shows genotype counts and allelic and genotypic association test results for all 84 SNPs.

FIG. 10 shows genotype counts and mean allelic and genotypic test results in the 10 imputed datasets.

FIG. 11 shows results using alternative approaches for SNP selection. Analyses are summarized for a) a stepwise search using the original data but different starting SNPs, b) analyses of the 11 imputed datasets each starting with the SNP showing the strongest association in the imputed data, and c) analysis of a dataset where the most likely genotype was imputed at each position and a stepwise logistic regression procedure to select associated SNPs. In each row, a likelihood ratio test (LRT) statistic comparing haplotype frequencies for the selected SNPs between cases and controls is given. The statistic was calculated using FUGUECC. In the case of the imputed datasets, the statistic was calculated after filling in the missing genotypes.

FIG. 12 shows (A) results of exhaustive search for the best SNP combination. All combinations of 1, 2, 3, 4 and 5 SNPs (˜33 million SNP combinations examined) were searched for the best associated SNPs and results are summarized in the following table. LRT is the likelihood ratio test statistic obtained from FUGUE-CC using the selected SNPs. Case-control labels were then permuted and re-applied the exhaustive search procedure to identify the combination of SNPs associated with the largest LRT statistic. For 1-4 SNPs, 100 permuted datasets were analyzed. For 5 SNPs, only 10 permuted datasets were analyzed. The results are summarized in (B). Note that in the permuted datasets each additional SNP increases the LRT by ˜10-15 units, whereas in the original dataset the 2nd, 3rd, 4th and 5th SNP increased the LRT by 86.82, 48.66, 45.19 and 20.93 units respectively.

FIG. 13 shows haplo-genotype counts for cases and controls. The table summarizes estimated counts for the identified haplotypes. The counts were estimated after using PHASE to haplotype all 84 SNPs simultaneously h1 to h8 represent the 8 haplotypes listed in FIG. 4.

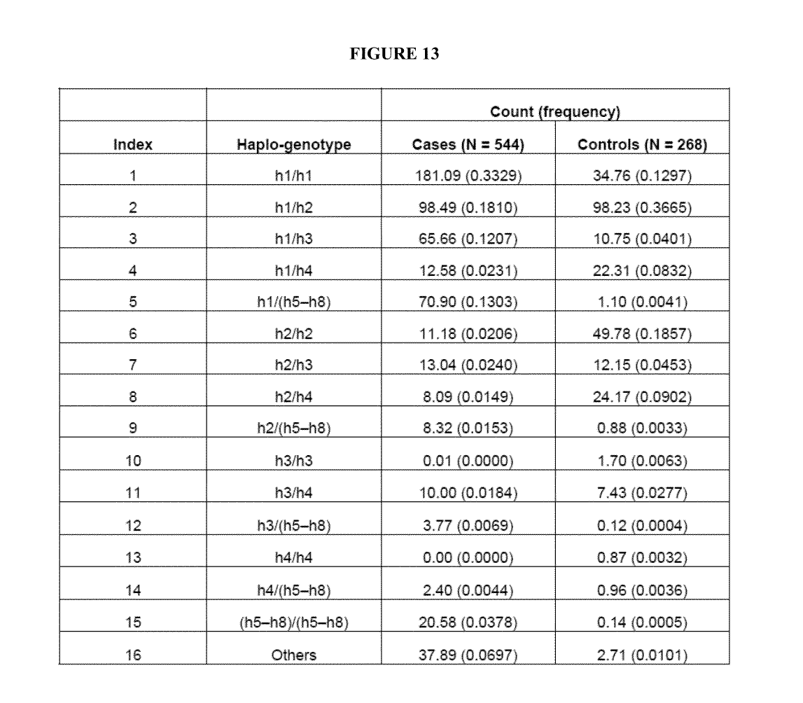

FIG. 14 shows association analysis of the 10q26 chromosomal region. P values for single SNP association tests comparing unrelated cases and controls. The genes in the indicated region are PLEKHA1, LOC387715/ARMS2,HTRA1 and DMBT1. rs10490924, the SNP showing strongest association in the region, is colored in red. Markers in strong association are colored in blue (r²>0.5) or green (r²>0.3).

FIG. 15 shows a graphical overview of linkage disequilibrium among 45 SNPs. The plot summarizes the linkage disequilbrium (D′) between all pairs of SNPs in the region (SNPs showing strong linkage disequilibrium (˜0.70 or greater), Intermediate levels of disequilibrium (˜0.30-0.70) and lower levels are shown.

FIG. 16 shows SNPs showing the strongest association with AMD. For each SNP, the risk allele(−) is defined as the allele with increased frequency in affected individuals. Evidence for association, as evaluated by the LAMP program (See Li M, Atmaca-Sonmez P, Othman M, Branham K E, Khanna R, Wade M S, Li Y, Liang L, Zareparsi S, Swaroop A, et al. (2006) Nat Genet 38; 1049-1054), is summarized through the risk allele frequency in the population (estimated using a parametric model that, in effect, weights cases and controls according to the estimated disease prevalence), LOD score (log₁₀ likelihood-ratio statistic comparing model with and without association), P value, and a series of estimated penetrances for non-risk homozygotes(+/+), heterozygotes(+/−) and risk allele homozygotes(−/−), genotype relative risks RR1 and RR2 (which are computed by comparing estimated penetrances in heterozygotes and risk-allele homozygotes, respectively, and those for non-risk homozygotes) and sibling recurrence risks λ_{sib}. The λ_{sib} measure characterizes the overall contribution of a locus to disease susceptibility. It quantifies the increase in risk to siblings of affected individuals attributable to a specific locus (See Risch N (1990) Am J Hum Genet 46; 222-228). For example, λ_{sib} of 1.27 signifies the SNP could account for a 27% in risk of AMD for relatives of affected individuals. Association analysis using a simple chi-squared statistic produced similar results. The last two columns summarize p-value results of logistic regression analysis including either rs10490924 or rs11200638 as covariates. Missing genotypes were imputed prior to the sequential analyses reported in the last two columns.

FIG. 17 shows chromosome 10q26 SNPs showing the association with AMD susceptibility. Single SNP association results are provided for all 45 markers. The rs number for each SNP is followed by the risk allele (the allele with higher frequency in affected individuals than in controls). Parametric association analyses were performed with the LAMP program (See Li M, Boehnke M, & Abecasis G R (2005) Am J Hum Genet 76; 934-949), which uses maximum likelihood to estimate a multiplicative disease model at each SNP (consisting of disease allele frequency and relative risk). The frequency of the risk allele in the population, penetrance for each genotype, the sibling recurrence risk λ_{sib}, and relative risks are also tabulated.

FIG. 18 shows observed allele counts and genomic context for each of the SNPs examined. The ‘−’ allele corresponds to the risk allele indicated in FIG. 17. N is the number of genotypes available among unrelated individuals; LRT is the standard likelihood ratio test statistic that is used to compare allele frequencies in cases and controls.

FIG. 19 shows linkage disequilibrium (LD) coefficients (D′, top, r₂, bottom) for all marker pairs examined. LD coefficients were estimated using an E-M algorithm implemented in the GOLD package (See Abecasis G R & Cookson W O (2000) Bioinformatics 16; 182-183).

FIG. 20 shows an analysis of the HTRA1 promoter region and AMD-associated SNP rs11200638. (A) Schematic representation of the human and mouse HTRA1 upstream promoter region and of luciferase reporter constructs used in the transactivation assays. The gray boxes indicate the genomic regions conserved between human and mouse, and the arrow indicates the position of rs11200638 SNP. HTRA1 promoter fragments (L-3.7 kb, M-0.83 kb, and S-0.48 kb) were cloned into pGL3-basic plasmid with the luciferase reporter gene. (B) Three different lengths of HTRA1 WT promoter-luciferase constructs (WT-L, -M, and -S) and two mutant constructs (SNP-L and -M) were transfected into HEK293 cells. Promoterless vector, pGL3, was used as a negative control, and the value of luciferase activity was set to 1. (C) and (D) are same as (B), except that ARPE-19 or Y79 cells were transfected with the promoter constructs. (E) Sequence comparison between human and mouse HTRA1 upstream promoter region spanning rs11200638 (gray box) using rVISTA (See Loots G G, Ovcharenko I, Pachter L, Dubchak I, & Rubin E M (2002) Genome Res 12; 832-839.). Predicted transcription factor binding sites are shown. The bold line indicates the oligonucleotide that was used as a probe for electrophoretic mobility shift assays (EMSA). (F) EMSA for rs11200638 spanning region. The [³²P]-labeled WT (lanes 1-6, and 10) or SNP (lanes 7-8) oligonucleotide probe was incubated with bovine retina nuclear extracts (BRNE). Competition experiments were performed with the unlabeled 50× specific (lane 3) or 50− non-specific (lane 4) oligonucleotide to validate the specificity of the band shift. EMSA experiments were also performed in the presence of the antibody against activating enhancer-binding protein-2α (AP-2α) (lanes 5 and 8), stimulating protein 1 (SP-1) (lanes 6 and 9), and neural retina leucine zipper protein (NRL) (lane 10). NRL antibody represents a negative control. The arrow shows the position of a specific DNA-protein binding complex.

FIG. 21 shows amino acid sequence and expression of the LOC387715/ARMS2 protein. (A) Amino acid sequence alignment and secondary structure analysis. Line 1: Amino acid sequence of the predicted human LOC387715/ARMS2 protein. Line 2: chimpanzee LOC387715/ARMS2 sequence. Line 3: Wild-type LOC387715/ARMS2 secondary structure prediction: H=helix, E=strand, C=the rest. Line 4: Secondary structure of LOC387715/ARMS2 altered by the A69S variation: Dot=same as WT. The gray box shows Ala codon 69 that is altered by the SNP rs10490924. (B) RT-PCR analysis of LOC387715/ARMS2 transcripts in cultured cell lines and in the retina of control and AMD subjects. HPRT was used as a control to evaluate RNA quality and normalize for the quantity. All PCR products were confirmed by sequencing. (C) Immunoblot analysis of COS-1 whole cell extracts, expressing human LOC387715/ARMS2 protein with N-terminal Xpress-tag. The expressed LOC387715/ARMS2 protein was detected using anti-LOC387715/ARMS2 (anti-LOC) or anti-Xpress (anti-Xp) antibody. (D) Fractionation of COS-1 cell extracts expressing LOC387715/ARMS2. Un+Nu, unbroken cells and nuclear fraction; Mt, mitochondria fraction; Sol, soluble fraction. (E) Proteinase K treatment of the mitochondria. The mitochondrial fractions from transfected COS-1 were treated with increasing concentrations of Proteinase K (ProK). The antibodies used for immunoblot analysis are indicated.

FIG. 22 shows subcellular localization of the LOC387715/ARMS2 protein. Human LOC387715/ARMS2 cDNA was cloned in pcDNA4 vector and transiently expressed in COS-1 cells. The cells were stained with anti-Xpress and an organelle-specific marker: (A) MitoTracker and (B) anti-COX IV antibody for mitochondria; (C) anti-PDI antibody for endoplasmic reticulum; (D) anti-Giantin antibody for Golgi; and (E) LysoTracker for lysosome. Bisbenzimide was used to stain the nuclei. Scale bar, 25 μm.

FIG. 23 shows primers for 10q26 SNPs that were PCR-amplified and sequenced.

FIG. 24 shows primer and oligonucleotide probe sequences.

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having AMD” refers to a subject that presents one or more symptoms indicative of age-related macular degeneration or is being screened for AMD (e.g., during a routine physical). A subject suspected of having AMD may also have one or more risk factors. A subject suspected of having AMD has generally not been tested for AMD. However, a “subject suspected of having AMD” encompasses an individual who has received a preliminary diagnosis but for whom a confirmatory test has not been done. A “subject suspected of having AMD” is sometimes diagnosed with AMD and is sometimes found to not have AMD.

As used herein, the term “subject diagnosed with a AMD” refers to a subject who has been tested and found to have cancerous cells. AMD may be diagnosed using any suitable method, including but not limited to, the diagnostic methods of the present invention.

As used herein, the term “initial diagnosis” refers to a test result of initial AMD diagnosis that reveals the presence or absence of AMD. An initial diagnosis does not include information about the stage or extent of AMD.

As used herein, the term “subject at risk for AMD” refers to a subject with one or more risk factors for developing AMD. Risk factors include, but are not limited to, gender, age, genetic predisposition, environmental exposure, and lifestyle.

As used herein, the term “characterizing AMD in subject” refers to the identification of one or more properties of AMD in a subject. AMD may be characterized by the identification of one or more markers (e.g., SNPs and/or haplotypes) of the present invention.

As used herein, the term “reagent(s) capable of specifically detecting biomarker expression” refers to reagents used to detect the expression of biomarkers (e.g., SNPs and/or haplotypes described herein). Examples of suitable reagents include but are not limited to, nucleic acid probes capable of specifically hybridizing to mRNA or cDNA, and antibodies (e.g., monoclonal antibodies).

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “providing a prognosis” refers to providing information regarding the impact of the presence of AMD (e.g., as determined by the diagnostic methods of the present invention) on a subject's future health.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5 +0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/1 EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-)specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (Kacian et al., Proc. Natl. Acad. Sci. USA 69:3038 (1972)). Other nucleic acids will not be replicated by this amplification enzyme. Similarly, in the case of T7 RNA polymerase, this amplification enzyme has a stringent specificity for its own promoters (Chamberlin et al., Nature 228:227 (1970)). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989)).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target.” In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]), has been modified by several groups to optimize conditions for particular types of cells. The art is well aware of these numerous modifications.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻0 cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates generally to biomarkers for macular degeneration. In particular, the present invention provides a plurality of biomarkers (e.g., polymorphisms and/or haplotypes) for monitoring and diagnosing macular degeneration. The compositions and methods of the present invention find use in diagnostic, therapeutic, research, and drug screening applications. The present invention further provides assay for identifying, characterizing, and testing therapeutic agents that find use in treating macular degeneration.

Accordingly, in some embodiments of the invention, experiments were conducted during development of embodiments of the invention to ascertain the impact of 84 polymorphisms in a region of 123 kb overlapping CFH on disease susceptibility.

As described herein, and in some embodiments of the present invention, the present invention provides (i) multiple variants show stronger association with AMD than the Y402H polymorphism, (ii) variants showing the strongest association appear to effect no change in the CFH protein, (iii) multiple haplotypes in the region modulate risk of AMD, and (iv) there are multiple disease-predisposing variants in the region.

Although an understanding of the mechanism is not necessary to practice the present invention and the present invention is not limited to any particular mechanism of action, in some embodiments, associated variants (or haplotypes) modulate risk of AMD not because they disrupt CFH protein function, but because they are important for regulating the expression of CFH, of other nearby complement genes or both (the region includes numerous CFH-like genes with similar sequences whose presence may account, in part, for the many SNPs in public databases for which a successful genotyping assays could not be executed; See, e.g., Methods described herein). Using genotypes for the HapMap panel of individuals²⁴ and gene expression data for 37 lymphoblastoid cell lines²⁵, the effect of the 84 SNPs examined herein was evaluated for the expression of transcripts in the CFH cluster in leukocytes. After Bonferroni adjustment for multiple testing, no evidence for association (P<0.05) was found.

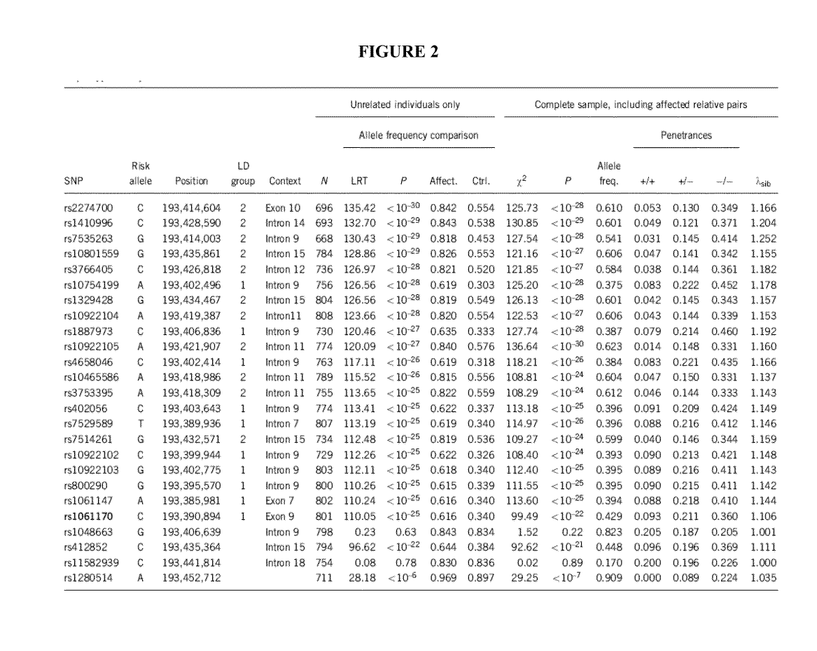

In some embodiments, the present invention provides the characterization of additional susceptibility alleles at the CFH locus, and provides that, even if the Y402H variant plays a causal role in the etiology of AMD, it is unlikely to be the only major determinant of disease susceptibility in the region. Indeed, the present invention identifies multiple other determinants of disease susceptibility (See FIGS. 2-5). Although an understanding of the mechanism is not necessary to practice the present invention and the present invention is not limited to any particular mechanism of action, in some embodiments, it is possible that Y402H is simply in linkage disequilibrium (LD) with nearby alleles that show even stronger association. In some embodiments, a strong LD in the region means that statistical methods will have limited resolution to distinguish between alternative sets of strongly associated SNPs. Accordingly, embodiments of the present invention contemplates detailed sequence comparisons of the region encompassing CFH in affected and unaffected individuals, examination of individuals from populations that show less extensive LD and dissection of gene expression patterns in individuals carrying different CFH haplotypes.

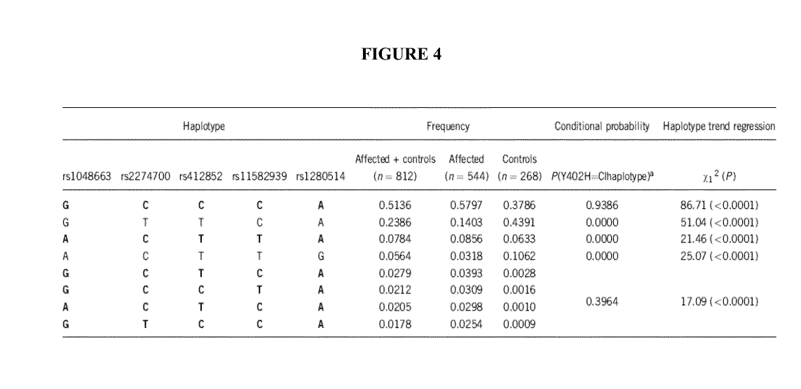

Prior to the development of the present invention, a common polymorphism encoding the sequence variation Y402H in CFH served as one of the only markers for susceptibility to age-related macular degeneration (AMD). However, experiments conducted during embodiments of the present invention have identified, in addition to the Y402H variation, 4-5 SNPs that are required to describe association between the CFH locus and AMD susceptibility. In particular, embodiments of the present invention provide four common haplotypes that can be used to diagnose susceptibility to AMD. For example, the present invention provides details of haplotypes defined by the five selected SNPs and their frequencies in affected individuals and controls (See FIG. 4). The present invention provides two common disease susceptibility haplotypes, two common protective haplotypes, and a set of rare haplotypes, which in the aggregate are associated with increased disease susceptibility. The C allele of Y402H was present in ˜94% of chromosomes that carry the most common risk haplotype and was absent from the common protective haplotypes. However, the allele was also absent from chromosomes carrying the second common risk haplotype (See FIG. 4). Thus, embodiments of the present invention provide that on its own, neither Y402H nor any of the other 83 variants examined could distinguish the common risk haplotypes from the common protective haplotypes. In addition, a combination of alleles at two or more SNPs that was shared between the two common risk haplotypes but absent from the protective haplotypes (or vice versa) were not identified. Thus, embodiments of the present invention provide that there are multiple susceptibility alleles in the region.

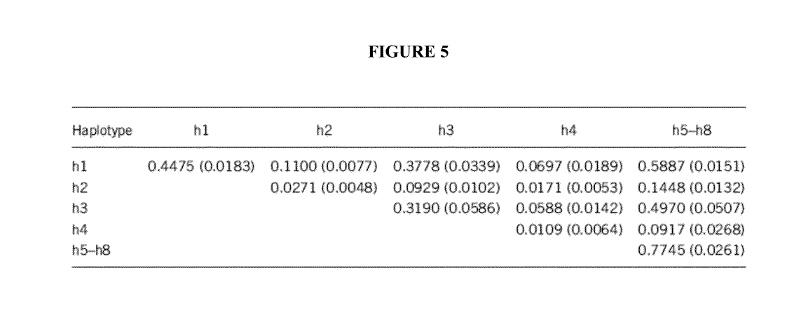

In some embodiments, the present invention further provides that inspection of genotype frequencies in affected individuals and controls provides that individuals carrying zero, one or two risk haplotypes are at progressively increased risk of developing disease. For example, FIG. 5 presents the estimated probability of disease for each possible haplo-genotype combination.

Thus, in some embodiments, the present invention provides different subsets of markers (e.g., biomarkers (e.g., alleles)) that can be used to distinguish risk and non-risk haplotypes for AMD. In some embodiments, risk or non-risk for AMD susceptibility is determined by detecting one or more sequences (e.g., alleles, SNPs, polymorphisms, variants, and/or haplotypes) described herein. In some embodiments, risk or non-risk for AMD susceptibility is determined by detecting sequences (e.g., SNPs, polymorphisms, variants, and/or alleles) that are in linkage disequilibrium with the SNPs described herein (e.g., those that are correlated to greater than 60%, 70%, 75%, 80%, 85%, 90%, 95%, 97%, 98% or more with the SNPs described herein).

Accordingly, in some embodiments, the present invention provides methods for detection of AMD and/or methods for diagnosing a subject's susceptibility for AMD. In some embodiments, the present invention detects the presence of one or more of the SNPs described herein. The present invention is not limited by the method utilized for detection. Indeed, a variety of different methods are known to those of skill in the art including, but not limited to, microarray detection, TAQMAN, PCR, allele specific PCR, sequencing, and other methods.

In some embodiments, the present invention provides kits for the detection and characterization of AMD. In some embodiments, the kits contain reagents for detecting SNPs described herein and/or antibodies specific for AMD biomarkers, in addition to detection reagents and buffers. In other embodiments, the kits contain reagents specific for the detection of AMD biomarker mRNA, SNPs, cDNA (e.g., oligonucleotide probes or primers), etc. In preferred embodiments, the kits contain all of the components necessary to perform a detection assay, including all controls, directions for performing assays, and any necessary software for analysis and presentation of results.

In some embodiments, the expression of mRNA and/or proteins associated with SNPs of the present invention are determined. In some embodiments, the presence or absence of SNPs are correlated with mRNA and/or protein expression. In some embodiments, gene silencing (e.g., siRNA and/or RNAi) is utilized to alter expression of genes associated with SNPs described herein.

In some embodiments, the present invention provides that rs10490924 SNP alone, or a variant in strong linkage disequilibrium therewith, is responsible for the association between the 10q26 chromosomal region and AMD. In some embodiments, the present invention provides that a previously-suggested causal SNP, rs11200638, and other examined SNPs in the region are indirectly associated with AMD. Thus, in some embodiments, and contrary to previous reports, the present invention provides that rs11200638 SNP has no significant impact on HTRA1 promoter activity in three different cell lines, and HTRA1 mRNA expression exhibits no significant change between control and AMD retinas. The present invention provides that SNP rs10490924 shows the strongest association with AMD (P=5.3*10⁻³⁰ ), and identifies an estimated relative risk of 2.66 for GT heterozygotes and 7.05 for TT homozygotes.

In some embodiments, the present invention provides that the rs10490924 SNP results in nonsynonymous A69S alteration in the predicted protein LOC387715/ARMS2, which has a highly-conserved ortholog in chimpanzee but not in other vertebrate sequences. Moreover, in some embodiments, the present invention provides that LOC387715/ARMS2 mRNA is present in the human retina and various cell lines and that it encodes a 12 kDa protein that localizes to the mitochondrial outer membrane when expressed in mammalian cells. The present invention provides that rs10490924 represents a major causal susceptibility variant for AMD at 10q26. Although an understanding of the mechanism is not necessary to practice the present invention, and the present invention is not limited to any particular mechanism, in some embodiments, the present invention provides that the A69S change in the LOC387715/ARMS2 protein affects the protein's function in mitochondria.

Experiments conducted during development of embodiments of the present invention clarify the genetic association with AMD and evaluate possible mechanism(s) of disease susceptibility. Since SNPs showing the strongest association alter the predicted coding sequence of LOC387715/ARMS2 and are upstream of HTRA1/PRSS11, experiments were conducted to investigate the biological function of LOC387715/ARMS2 and examine the previously-proposed impact of rs11200638 on the expression of HTRA1/PRSS11. The present invention provides a direct comparison of HTRA1 and LOC387715/ARMS2 SNPs and provides that a single variant of large effect exists in the region. Specifically, after examining a set of SNPs that tags common variants in the region, the strongest association was with rs10490924, a SNP that affects the coding sequence of LOC387715/ARMS2 (P<10⁻²⁹) (See Example 3). Evidence for association is weaker at all other SNPs (P>10⁻²¹) and becomes non-significant after accounting for rs10490924 in a multiple regression analysis.

The present invention provides that rs10490924 alters the predicted coding sequence of LOC387715/ARMS2. LOC387715/ARMS2 is listed as a hypothetical human gene with highly-conserved ortholog in chimpanzee, but not in sequences from other organisms. The two exons of LOC387715/ARMS2 encode a putative protein of 107 amino acids, which includes no remarkable motifs, except for nine predicted phosphorylation sites. The present invention identifies the presence of LOC387715/ARMS2 transcripts in human retina and variety of other tissues and cell lines. Furthermore, the present invention provides the translatation of LOC387715/ARMS2 cDNA cloned from the human retina, demonstrating that LOC387715/ARMS2 encodes a bona-fide protein.

Although an understanding of the mechanism is not necessary to practice the present invention, and the present invention is not limited to any particular mechanism, in some embodiments, the present invention provides that localization of the LOC387715/ARMS2 protein to mitochondrial outer membrane in transfected mammalian cells provides a mechanism through which A69S change can influence AMD susceptibility. For example, mitochondria are implicated in the pathogenesis of age-related neurodegenerative diseases, including Alzheimer's disease, Parkinson's disease and Amyotrophic lateral sclerosis (See, e.g., Lin M T & Beal M F (2006) Nature 443; 787-795). Mitochondrial dysfunction associated with aging can result in impairment of energy metabolism and homeostasis, generation of reactive oxygen species, accumulation of somatic mutations in mitochondrial DNA, and activation of the apoptotic pathway (See, e.g., Lin M T & Beal M F (2006) Nature 443; 787-795; Kroemer G & Reed J C (2000) Nat Med 6; 513-519; Barron M J, Johnson M A, Andrews R M, Clarke M P, Griffiths P G, Bristow E, He L P, Durham S, & Turnbull D M (2001) Invest Ophthalmol Vis Sci 42; 3016-3022; Wright A F, Jacobson S G, Cideciyan A V, Roman A J, Shu X, Vlachantoni D, McInnes R R, & Riemersma R A (2004) Nat Genet 36; 1153-1158; Wallace D C (2005) Annu Rev Genet 39; 359-407; McBride H M, Neuspiel M, & Wasiak S (2006) Curr Biol 16; R551-560; Feher J, Kovacs I, Artico M, Cavallotti C, Papale A, & Balacco Gabrieli C (2006) Neurobiol Aging 27; 983-993). Decreased number and size of mitochondria, loss of cristae or reduced matrix density are observed in AMD retina compared to control, and mitochondrial DNA deletions and cytochrome c oxidase-deficient cones accumulate in the aging retina, particularly in the macular region (See, e.g., Barron M J, Johnson M A, Andrews R M, Clarke M P, Griffiths P G, Bristow E, He L P, Durham S, & Turnbull D M (2001) Invest Ophthalmol Vis Sci 42; 3016-3022; Feher J, Kovacs I, Artico M, Cavallotti C, Papale A, & Balacco Gabrieli C (2006) Neurobiol Aging 27; 983-993). Moreover, mutations in mitochondrial proteins (e.g., dynamin-like GTPase OPA1) are associated with optic neurodegenerative disorders (See, e.g., Carelli V, Ross-Cisneros F N, & Sadun A A (2004) Prog Retin Eye Res 23; 53-89).

Photoreceptors and RPE contain high levels of polyunsaturated fatty acids and are exposed to intense light and near-arterial level of oxygen, providing considerable risk for oxidative damage. Thus, in some embodiments, the present invention provides that altered function of the putative mitochondrial protein LOC387715/ARMS2 by A69S substitution enhances the susceptibility to aging-associated degeneration of macular photoreceptors. Accordingly, the present invention also provides, in some embodiments, methods of identifying risk for AMD by characterizing LOC387715/ARMS2 in subject (e.g., characterizing the presence of or the absence of the A69S mutation or mutations in linkage with A69S, alone or together with one or more other biomarkers (e.g., SNPs) described herein or with one or more other markers of macular degeneration). In some embodiments, mutations that cause truncation of the ARMS2 protein (e.g., by introduction of an early stop codon) or large insertions or deletions are detected as correlated to aberrant ARMS2 protein, for example, having a detrimental impact on normal mitochondrial biology and an associated increase in risk of AMD. Experiments conducted during development of some embodiments of the present invention provide that there is not any significant difference in the expression, stability or localization of the A69S variant LOC387715/ARMS2 protein in mammalian cells. Thus, in some embodiments, the present invention provides that the A69S alteration modifies the function of LOC387715/ARMS2 protein by affecting its conformation and/or interaction.

In some embodiments, the present invention contemplates screening arrays of compounds (e.g., pharmaceuticals, drugs, peptides, or other test compounds) for their ability to alter LOC387715/ARMS2 protein (e.g., alter its conformation and/or interaction with other proteins) or to compensate for altered ARMS2 function. In some embodiments, compounds (e.g., pharmaceuticals, drugs, peptides, or other test compounds) identified using screening assays of the present invention find use in the treatment of AMD (e.g., although a mechanism is not necessary to practice the present invention and the present invention is not limited to any particular mechanism, in some embodiments, a compound so identified stabilizes LOC387715/ARMS2 protein conformation and/or its interaction with other proteins).

In some embodiments, the present invention provides a method to assay the effects of ARMS2, and variants thereof, on mitochondria. In some embodiments, the ARMS2 gene, and/or variants thereof, are stably integrated into the genomes of non-human animals (e.g. mice, rats, etc.) to create animal lines expressing the ARMS2 gene or variants thereof. In some embodiments, variants of ARMS2 may contain, but are not limited to insertions, deletions, insertion-deletions, substitutions, etc. In some embodiments, the non-human animal lines with stably integrated ARMS2, and variants thereof, can serve as ARMS2 and variant ARMS2 animal models. In some embodiments, the non-human animal lines with stably integrated ARMS2, and variants thereof, can serve as animal models to compare ARMS2, and variant ARMS2 function. In some embodiments, cell lines can be produced containing ARMS2 and variants thereof. In some embodiments, variants of ARMS2 integrated into cell lines may include, but are not limited to insertions, deletions, insertion-deletions, substitutions, single nucleotide polymorphisms, etc. In some embodiments, cell lines produced containing ARMS2, and variants thereof, can serve as ARMS2, and variant ARMS2, cell culture models. In some embodiments, cell lines produced containing ARMS2, and variants thereof, can serve as cell culture models for ARMS2, and variant ARMS2, function. In some embodiments, ARMS2, and variant ARMS2, animal models and cell culture models of can be used to assay the effects that variants of ARMS2 have on mitochondrial function, output, health, etc. In some embodiments, ARMS2, and variant ARMS2, animal models and cell culture models can be used to assay the effects of ARMS2 and variant ARMS2 on the whole cell or organism.

In some embodiments, ARMS2, and variant ARMS2, animal models and cell culture models can be used to assay mitochondrial functions and characteristics including, but not limited to red-ox state, metabolism, fatty acid oxidation, glycolysis, oxidative stress, DNA oxidation, protein modification, lipoxidation, etc, and the effects of ARMS2 variants on the aforementioned mitochondrial functions and characteristics.

In some embodiments, the present invention provides screening assays for assessing cellular (e.g., mitochondrial) behavior or function. For example, the response of cells, tissues, or organisms to interventions (e.g., drugs, diets, aging, etc.) may be monitored by assessing, for example, mitrochondrial functions using animal or cell culture models as describe herein. Such assays find particular use for characterizing, identifying, validating, selecting, optimizing, or monitoring the effects of agents (e.g., small molecule-, peptide-, antibody-, nucleic acid-based drugs, etc.) that find use in treating or preventing macular degeneration or related diseases or conditions.

### EXPERIMENTAL

The following examples are provided in order to demonstrate and further illustrate certain preferred embodiments and aspects of the present invention and are not to be construed as limiting the scope thereof.

### Example 1

### Materials and Methods

### Subjects.

Families with AMD were primarily ascertained and recruited from the clinical practice at the Kellogg Eye Center, University of Michigan Hospitals. The patient population used for genotyping was white and primarily of Western European ancestry, reflecting the genetic constitution of the Great Lakes region. Ophthalmic records for current and previous eye examinations, fundus photographs and fluorescein angiograms were obtained for all probands and family members. All records and ophthalmic documentation were scored for the presence of AMD clinical findings in each eye and were updated every 1-2 years. The recruitment and research protocols were reviewed and approved by the University of Michigan institutional review board, and informed consent was obtained from all study participants. Fundus findings in each eye were classified on the basis of a standardized set of diagnostic criteria established by the International Age-Related Maculopathy Epidemiological Study²⁶. For the genetic studies described herein, macular findings were scored in each individual by use of a broad description of AMD. In total, a sample of 726 affected individuals included 235 affected relative pairs in 93 families (153 sibling pairs, 4 half-sibling pairs, 45 cousin pairs, 4 parent-child pairs and 29 avuncular pairs). Focusing on a subset of the sample that included only unrelated individuals resulted in 544 affected individuals and 268 unrelated controls.

### Genotyping and Quality Assessment.

A genotyping assays was designed for all 244 SNPs in the region (dbSNP 124, February 2005). Primers were successfully designed for 193 of these SNPs and genotyping was carried out on the Sequenom platform by the Broad Institute/National Center for Research Resources Genotyping Center (Cambridge, Mass.). To facilitate quality assessment, the 90 CEU samples that are part of the HapMap²⁴ were also genotyped. Coding SNPs where the initial genotyping assay failed were attempted through sequencing at the University of Michigan DNA Sequencing Core. Among the 193 SNPs for which assays were attempted, a total of 84 SNPs passed Hardy-Weinberg equilibrium (HWE) tests²⁷ (P>0.001), had >75% of genotypes completed and showed a minor allele frequency of >0.05. The 84 successfully assayed SNPs had average minor allele frequencies (MAF) of 0.281 and genotyping completeness rates of 93.17%. The remaining SNPs were excluded from further consideration because they were rare (46 SNPs had MAF <0.05) or monomorphic (25 SNPs), had low genotyping success rates (23 SNPs) or failed HWE (15 SNPs). The 23 SNPs with low completeness rates were excluded because missingness patterns suggested a high proportion of missing heterozygotes, consistent with limitations of the assay platform. For 42 SNPs, genotype calls were compared with those downloaded from the HapMap website and observed 15 discrepancies among 3,317 overlapping genotypes (genotyping error rate of ˜0.22%).

### Single-SNP Association Tests Comparing Unrelated Affected Individuals and Controls.

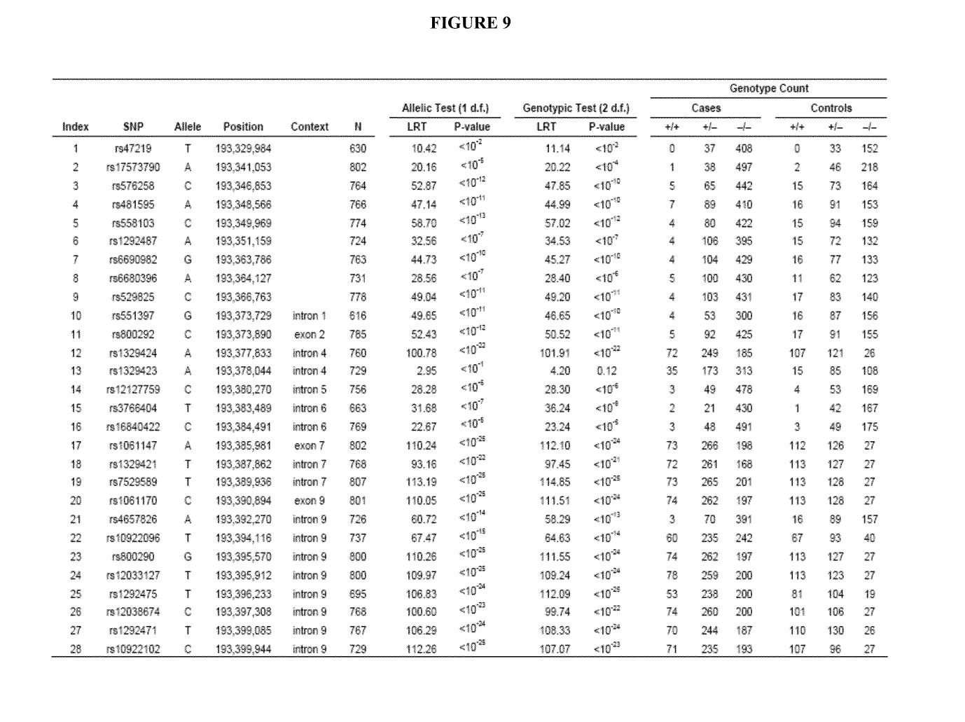

Allele frequencies in affected individuals and controls were compared using a standard likelihood ratio test statistic. Briefly, if the Oᵢⱼ denotes the observed counts for allele i (i=1 or 2) in group j (j=affected individuals or controls), and Eᵢⱼ denotes the expected counts under the null hypothesis of no association, then the test statistic was defined as χ²=2EᵢⱼOᵢⱼ ln Oᵢⱼ/Eᵢⱼ. Significance was evaluated against a reference χ² distribution with 1 degree of freedom. When we carried out a 2 d.f. association test (See FIG. 9), rankings for individual SNPs changed slightly but the top 10 SNPs remained the same in both the 1 d.f. and 2 d.f. analyses. When the 1 d.f. and 2 d.f. models were compared using logistic regression, no significant improvement in model fit from the 2 d.f. models was observed and thus the analysis presented herein focus on the 1 d.f. tests.

### Single-SNP Association Tests Incorporating Related Affected Individuals and Unrelated Controls.

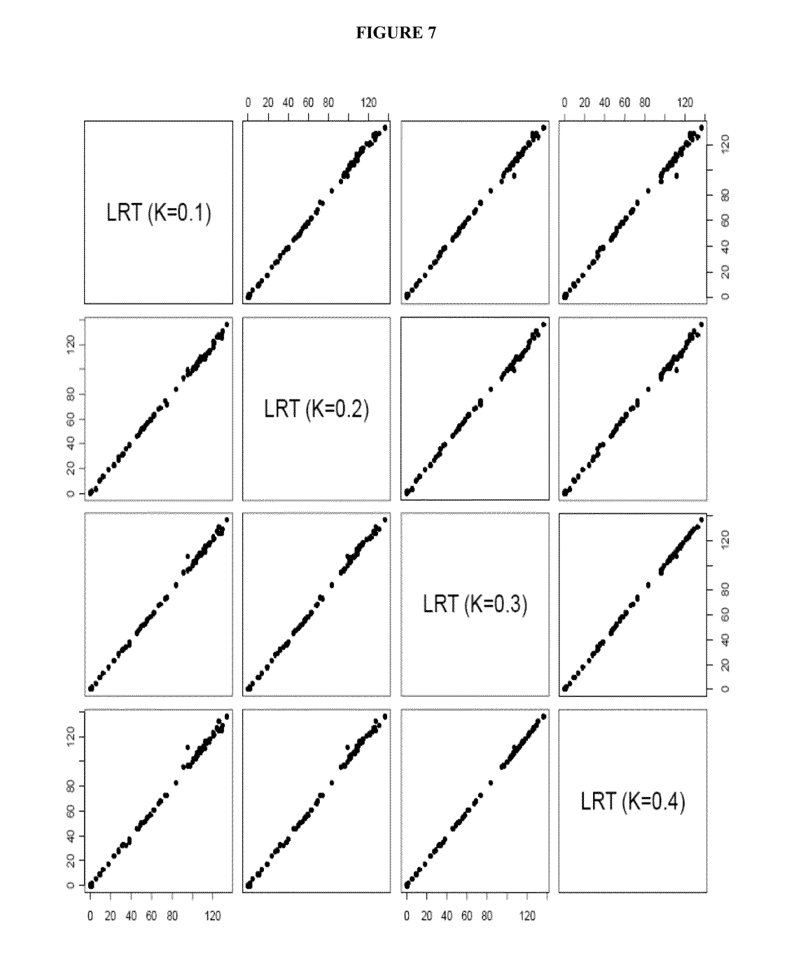

To incorporate all available genotype data in the test of association and to estimate genetic model parameters, parametric models of association were fitted using the LAMP^{16, 17} program. Briefly, the program estimates a disease allele frequency, a SNP allele frequency and three penetrances (constrained so that the disease prevalence=20%) using all available data. Each SNP was analyzed together with two flanking microsatellite markers (GATA135F02 and GATA48B01, genotyped as part of our genome-wide linkage scan²) and independently of all other SNPs. Under the null hypothesis (linkage but no association), the SNP and disease alleles are assumed to be in linkage equilibrium (this corresponds to calculating a MOD score¹⁹). Under the alternative hypothesis, LD between the SNP and unobserved disease alleles is estimated using maximum likelihood and results in a one-parameter test (because three disease-SNP haplotype frequencies are estimated under the alternative but only two allele frequencies are estimated under the null). The fitted model allows for ascertainment. The analyses assumed a fixed disease prevalence of 20%; different estimates would change parameter estimates, but do not affect the overall ranking of SNPs (See FIG. 7).

### Identification of Strongly Associated Haplotypes.

A stepwise procedure was used to identify the most strongly associated haplotypes. For each marker combination, haplotype frequencies in affected individuals, in controls and in the combined sample were estimated using maximum likelihood as implemented in FUGUE-CC²⁸. The three-frequency estimates were used to calculate the likelihood of observed case genotypes (L_{cases}), of observed control genotypes (L_{controls}) and of the combined set of genotypes (L_{combined}). A likelihood ratio statistic T=ln(L_{cases}L_{controls})−ln(L_{combined}) was used evaluate differences between cases and controls and its significance was evaluated by permuting case and control labels. At each stage, the marker producing the greatest increase in the test statistic T was added to the model. The significance of the improvement in model fit produced by adding the N^{th} marker by focusing on permutations that did not alter genotypes for the previously selected N−1 markers. This assessment of significance includes a built-in multiplicity adjustment, because at each stage the maximum observed test statistic from the original data was compare with the maximum statistics from the permuted datasets. The procedure is slightly conservative (that is, it slightly favors less complex models that include fewer SNPs), because the permutations become more and more constrained as additional SNPs are added into the model. However, given the large dataset and the presence of many common haplotypes, this concern is minor: even after selecting five SNPs, >10¹⁰⁵ distinct permutations of the data are possible. The permutation procedure described herein was used because it (i) naturally accommodates missing data (with 84 SNPs, many individuals have at least one missing genotype), (ii) preserves patterns of LD in the original data, (iii) allowed conditioning out of the effects of SNPs previously selected into the model and (iv) achieves a balance between a model that is too simple (for example, including only marginal effects) and one that is too complex (accounting for all genotype combinations). Individual haplotype effects were estimated using an approach analogous to one proposed previously by others²¹, but using logistic regression rather than linear regression to accommodate a discrete outcome.

### Stepwise Logistic Regression.

A stepwise-logistic regression was carried out using SAS version 9 (Cary, N.C.). Genotypes at each marker were coded as 0, 1 or 2, corresponding to a 1-d.f. test. Owing to strong LD in the region, when building the logistic regression model, the Wald test was not used, which is known to be unstable in the presence of collinearity. Rather, the log likelihoods of the nested models was compared using a likelihood ratio test. Similar to the stepwise haplotype analysis, at each stage, the marker producing the greatest increase in the LRT was added to the model (provided that adding the marker significantly improved the model, P<0.05).

### Electronic Database Information.

LAMP software for estimating MOD scores and fitting parametric association models in samples including unrelated individuals and/or family data is available online at http://www.sph.umich.edu/csg/abecasis/LAMP/.

### Example 2

### CFH Haplotypes without the Y402H Coding Variant Show Strong Association with Susceptibility to Age-Related Macular Degeneration

Experiments were conducted during development of embodiments of the invention to ascertain the impact of 84 polymorphisms in a region of 123 kb overlapping CFH on disease susceptibility.

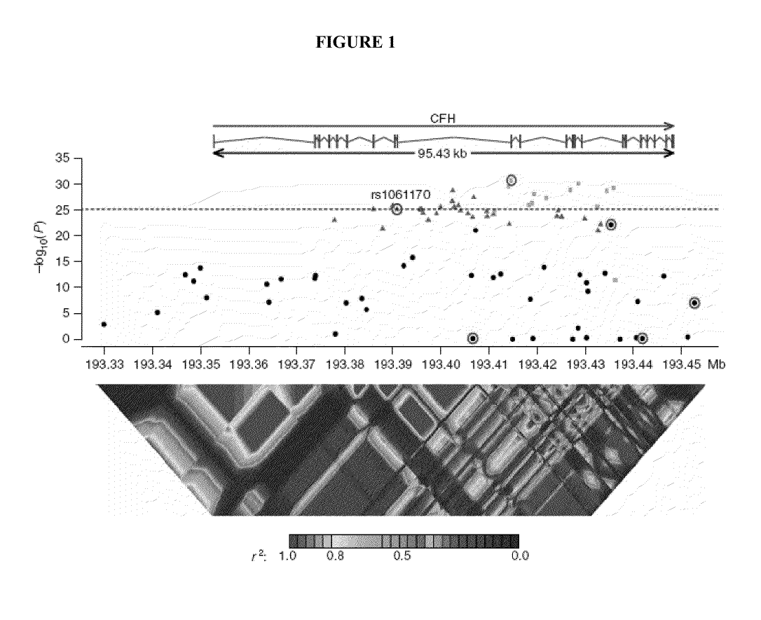

After quality assessment of genotype data (See Materials and Methods above), each SNP was tested for association in 544 unrelated affected individuals and 268 unrelated controls (See FIG. 1). A strong association was observed between disease status and the Y402H-encoding variant previously associated with AMD in multiple studies (likelihood ratio test χ²=110.05, P<10⁻²⁵). Unexpectedly, 20 other variants showed even stronger association. The strongly associated SNPs fell into two linkage disequilibrium (LD) groups (indicated as small triangles or small squares in FIG. 1), such that, within each group, pairwise r²>0.80, and between groups, pairwise r²<0.50. The Y402H-encoding variant was included in one of the LD groups (the triangle group in FIG. 1). The three SNPs showing strongest association are a synonymous SNP in exon 10, rs2274700 (LRT χ²=135.42, P<10⁻³⁰) and two intronic SNPs, rs1410996 (LRT χ²=132.70, P<10⁻²⁹) and rs7535263 (LRT χ²=130.43, P<10⁻²⁹). Similar results were observed using a family-based association test^{16, 17} that incorporated all 726 affected individuals genotyped.

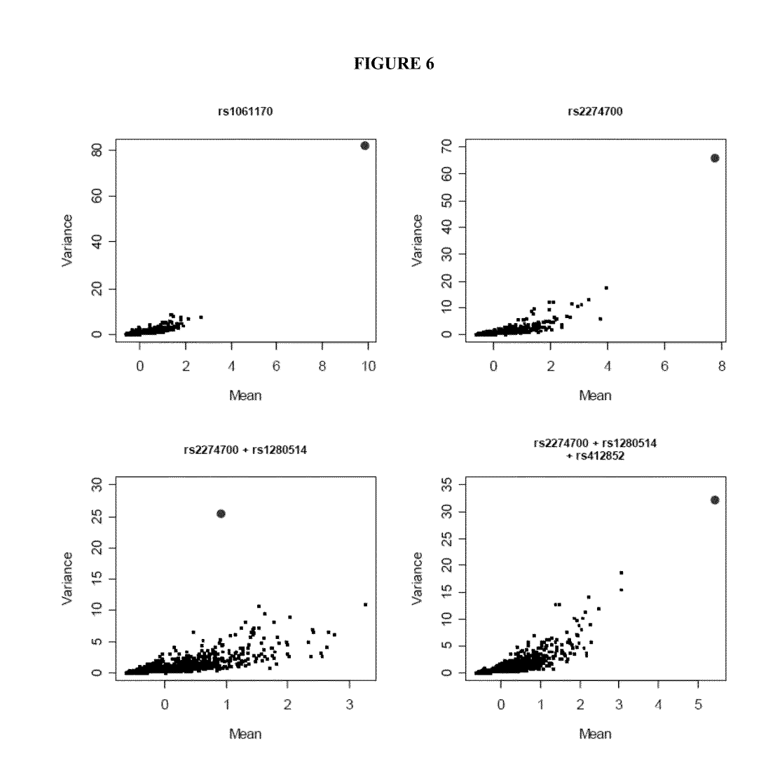

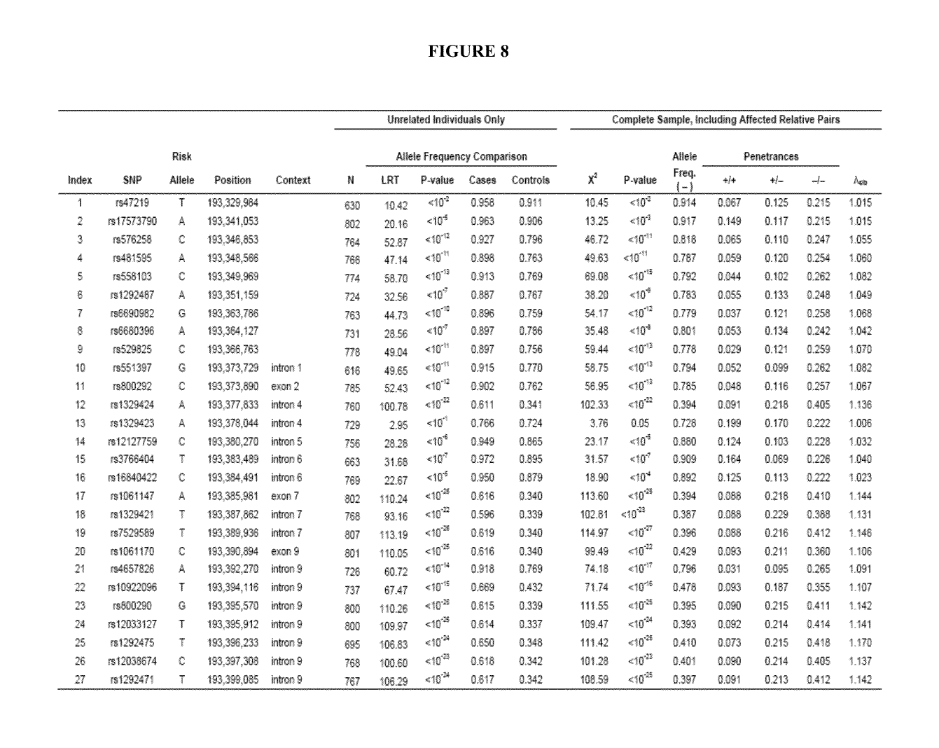

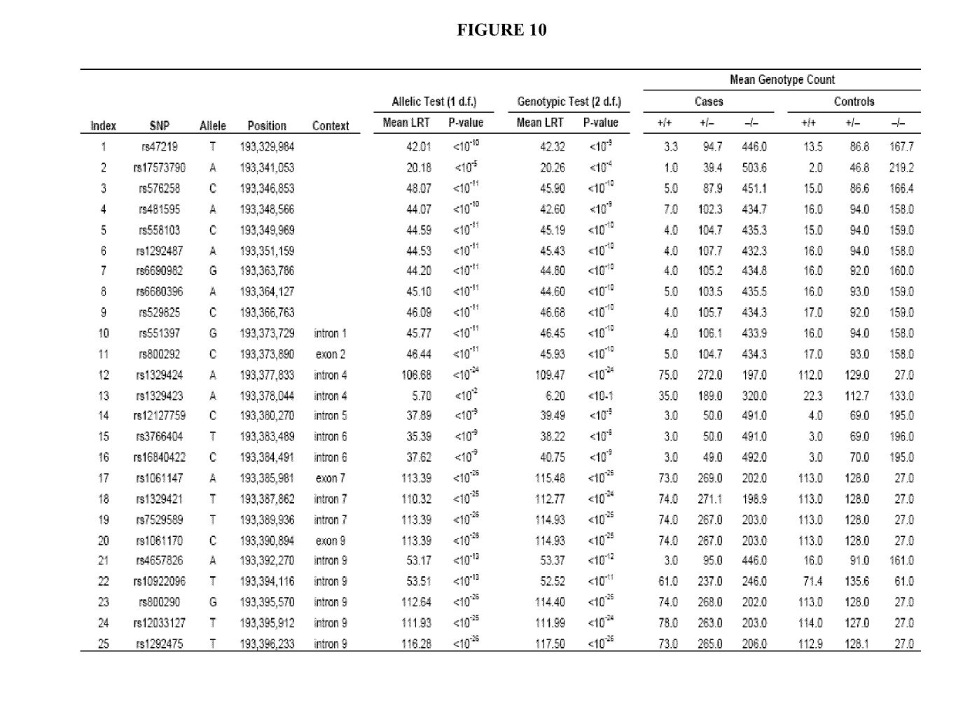

FIG. 2 summarizes results of family-based and case-control single-SNP association tests for rs1061170 (the Y402H coding polymorphism) and the 20 SNPs that showed even more significant association in the sample. FIG. 2 also includes four SNPs that showed weaker marginal association but that were included in the haplotype model detailed below. FIGS. 8-10 provide genotype counts and detailed results for all 84 SNPs (including 2 di. association test results). The estimated sibling recurrence risk ratio (λ_{sib}) (ref. 18) for rs1061170 is smaller than in previous analysis¹⁵, that had not accounted for the increased contrast between affected individuals and controls as a result of the selection of families with multiple affected individuals. In the present analysis, phenotypes were modeled for all affected individuals within each family simultaneously^{16, 17,} and it is expected that estimates of λ_{sib}, penetrances and allele frequencies are more accurate. To help interpret the λ_{sib} estimates associated with each polymorphism, previously genotyped microsatellite markers were also used to calculate a MOD score (LOD score maximized over mode of inheritance¹⁹) at the location of the CFH locus. The estimated MOD score was 1.76 (3 d.f., P=0.04) with an estimated disease allele frequency of 0.230 and penetrances of 0.044, 0.340 and 1.00 for low-risk allele homozygotes, heterozygotes and high-risk allele homozygotes, respectively. Notably, this disease model gave λ_{sib}˜1.67, but the largest λ_{sib} accounted for by a single SNP was only 1.25 (for marker rs7535263; see last column of FIG. 2). The haplotype method²⁰ also suggested the presence of multiple disease susceptibility alleles in the region, because haplotypes grouped according to either the allele encoding Y402H or the allele at rs2274700 (the marker showing strongest association) differed substantially between affected individuals and controls (See FIG. 6).

To further dissect the association between these polymorphisms and susceptibility to AMD, it was determined whether a model with two or more SNPs resulted in significantly stronger association. To do this, a likelihood ratio test (LRT) was used to compare haplotype frequencies between affected individuals and controls. The SNP showing the strongest association with disease was used first and then the model iteratively expanded one SNP at a time. At each iteration, the SNP that resulted in the largest increase in the LRT statistic was selected. The SNP that showed the strongest LRT association with disease was rs2274700 (LRT χ²=135.42, See FIG. 2). When evaluating all pairs of SNPs including rs2274700 and one other SNP, a very strong association was observed for haplotypes defined by pairing rs2274700 and rs1280514 (LRT χ²=188.69). To evaluate the statistical significance of this finding, case and control labels were permuted among individuals with the same genotype (C/C, C/T, T/T or missing) for marker rs2274700. This permutation preserves the LD pattern in the original sample as well as the association between rs2274700 and disease. For each permutation, the SNP pairing that produced the strongest association was selected and the increase recorded in the LRT statistic. In 10,000 permutations of the data, an average increase of 1.76 was observed in the LRT χ² statistic whereas an increase in the LRT χ²>53.27 was not observed, corresponding to the pairing of rs2274700 and rs1280514 in the original data.

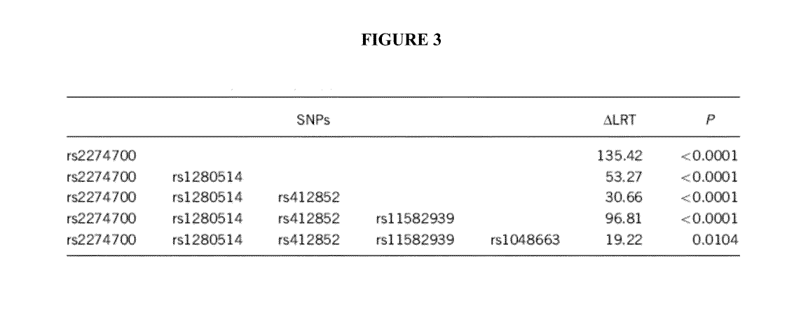

The haplotype model was refined in a similar manner. At each stage, the SNP producing the largest increase in the LRT χ₂ statistic was selected and empirical significance evaluated by permuting case and control labels among individuals with the same genotype at previously selected markers. FIG. 3 shows that 4-5 SNPs are required to describe association between the CFH locus and AMD susceptibility.

FIG. 4 provides details of haplotypes defined by the five selected SNPs and their frequencies in affected individuals and controls. Haplotype effects were estimated using logistic regression to model individual affection status as a function of the expected dosage of each haplotype²¹. Two common disease susceptibility haplotypes were identified, two common protective haplotypes were identified, and a set of rare haplotypes were identified, which in the aggregate appear to be associated with increased disease susceptibility. The C allele of Y402H was present in ˜94% of chromosomes that carry the most common risk haplotype and was absent from the common protective haplotypes. However, the allele was also absent from chromosomes carrying the second common risk haplotype (See FIG. 4). On its own, neither Y402H nor any of the other 83 variants examined could distinguish the common risk haplotypes from the common protective haplotypes. In addition, a combination of alleles at two or more SNPs that was shared between the two common risk haplotypes but absent from the protective haplotypes (or vice versa) were not identified. Thus, embodiments of the present invention provide that there are multiple susceptibility alleles in the region.

Inspection of genotype frequencies in affected individuals and controls provides that individuals carrying zero, one or two risk haplotypes are at progressively increased risk of developing disease. FIG. 5 presents the estimated probability of disease for each possible haplo-genotype combination, estimated using maximum likelihood and assuming disease prevalence of 20% and a multiplicative model for disease risk. Note that the estimated probabilities of developing disease for each genotype configuration depend on the overall disease prevalence, which varies with age.

Notably, when imputed haplotypes were recoded into a biallelic system (with a high-risk allele and a low risk allele), no evidence for additional linked variants^{16, 17} (LOD<0.01) were found. Further, using the haplotype method²⁰, haplotypes classified using the five selected markers were similar in affected individuals and controls (See FIG. 6). These two results provide that, if susceptibility alleles are not included in the set of genotyped variants, they will either be in very strong LD with the selected SNPs or have relatively small effects.

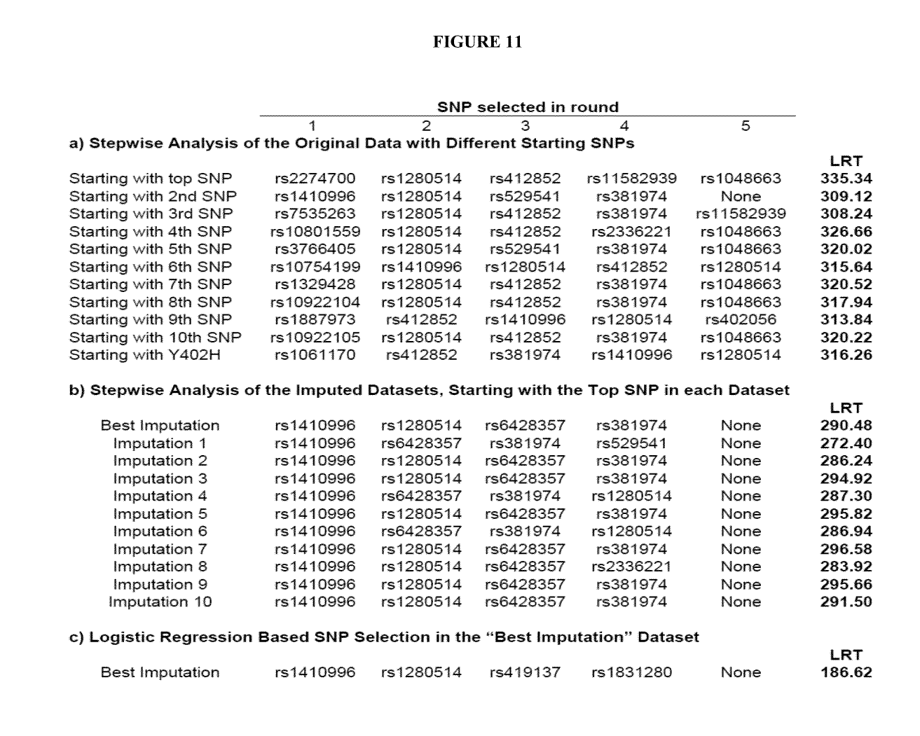

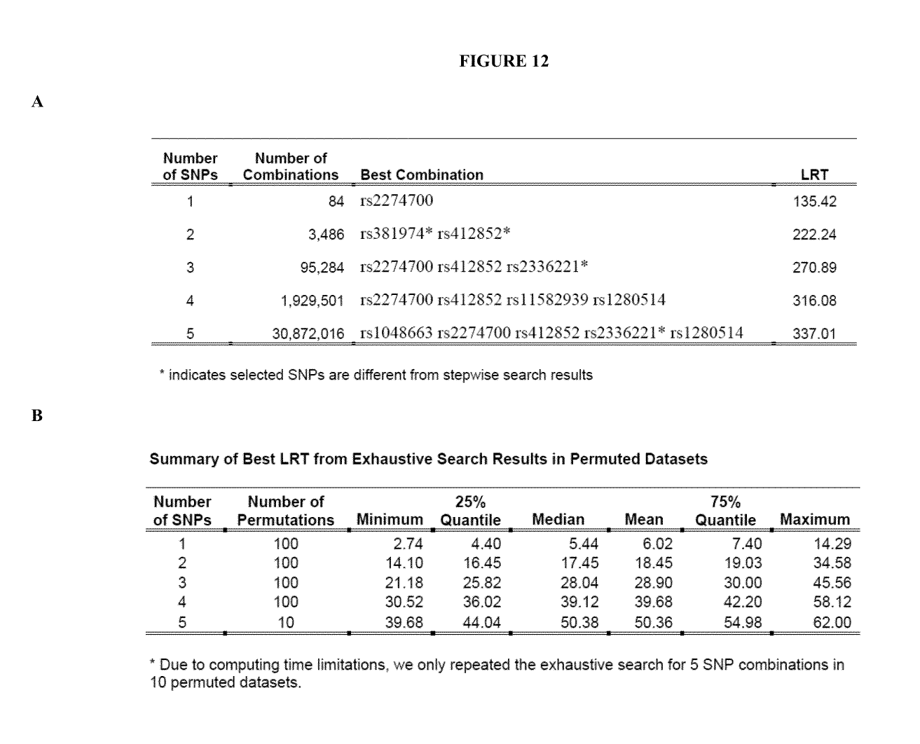

One concern is that the model selection procedure might affect the resulting set of risk and protective haplotypes and, ultimately, conclusions. Thus, the analysis was repeated using each of the ten SNPs showing the strongest evidence for association as the starting point for stepwise analysis. Depending on the choice of starting SNP, this resulted in a model with four or five SNPs (See FIG. 11). In each case, the selected SNPs were in strong LD with the originally selected SNPs. An exhaustive search procedure was also used to examine all possible combinations of up to five SNPs (See FIG. 12). The best four-SNP combination identified was the same as in the original stepwise analysis, and the best five-SNP combination differed by only one SNP (rs11582939 was replaced with rs2336221; r² between the two is >0.99). Given substantial LD in the region, it is not surprising that different subsets of markers can be used to distinguish risk and non-risk haplotypes. Nevertheless, in each of the alternative analyses, the selected SNPs defined two common risk haplotypes, two common protective haplotypes and a series of rare haplotypes that were, in the aggregate, most associated with disease.

Another possible concern is that vagaries of missing data patterns could strengthen or weaken the evidence of association for individual SNPs or haplotypes. To address this, PHASE^{22, 23} was used to impute missing genotypes. 3,372 (5%) of the available genotypes were initially masked to check the ability to infer the genotypes correctly. Only 33 mismatches were found between the original masked genotypes and inferred genotypes. Given the high quality of the inferred genotypes, the following were generated (i) a complete dataset by imputing the most likely genotype at each position using PHASE and (ii) ten additional datasets by sampling a plausible haplotype configuration for each individual, according to the posterior haplotype distribution estimated by PHASE. Single-marker and haplotype analyses were then repeated in each ‘completed’ dataset and stepwise logistic regression used to identify a set of associated SNPs in the best imputed dataset. In each case, the results were consistent with the initial analyses: multiple SNPs showed substantially stronger association than did Y402H, and the markers selected in haplotype analyses defined two common susceptibility haplotypes, two common protective haplotypes and multiple rare haplotypes associated with disease susceptibility in the aggregate (See FIG. 11).

### Example 3

### A Variant of Mitochondrial Protein LOC387715/ARMS2, Not HTRA1, is Strongly associated with Age-Related Macular Segeneration

### Materials and Methods

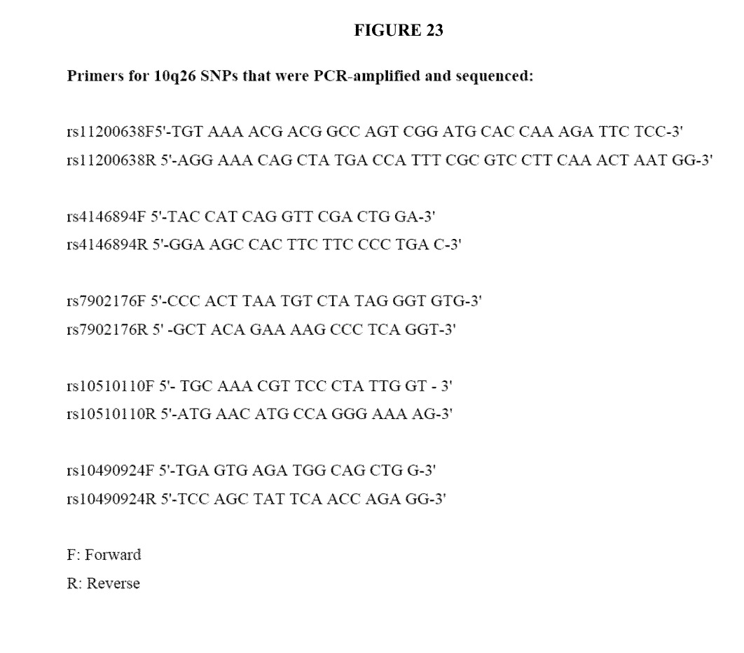

Genotyping and Data Analysis. Five hundred and thirty-five affected individuals and 288 unrelated controls were examined that were primarily ascertained and recruited at the Kellogg Eye Center, as described (See Zareparsi S, Branham K E, Li M, Shah S, Klein R J, Ott J, Hoh J, Abecasis G R, & Swaroop A (2005) Am J Hum Genet 77; 149-153; Li M, Atmaca-Sonmez P, Othman M, Branham K E, Khanna R, Wade M S, Li Y, Liang L, Zareparsi S, Swaroop A, et al. (2006) Nat Genet 38; 1049-1054). TaqMan assays (ordered from Applied Biosystems, Foster City, Calif.) were performed at the University of Michigan Sequencing Core Facility. For some SNPs (See FIG. 23), PCR was used for amplification prior to sequencing. In a follow-up experiment, a set of 20 overlapping markers (including rs10490924) were genotyped using an Illumina Golden Gate panel; a comparison to the original calls revealed an overall error rate of ˜1.0%, which did not differ between cases and controls. The Illumina genotypes (with an overall completeness of 98.9%) also provide much stronger association for rs10490924 than for any other marker in the region and that rs10490924 can explain observed results for all other SNPs. However, TAQMAN data is reported, despite the lower completeness, because it includes a larger number of SNPs in the region. Genotypes were checked for quality by examining call rates per marker and per individual and by calculating an exact Hardy-Weinberg test statistic (See Wigginton J E, Cutler D J, & Abecasis G R (2005) Am J Hum Genet 76; 887-893). After excluding individuals with <25 successfully-typed SNPs, a total of 280 controls and 466 cases were selected for analysis. The average genotyping completeness was 94.3%. Genotype frequencies between cases and controls were compared using a standard chi-squared tests and a model-based procedure (See Wigginton J E, Cutler D J, & Abecasis G R (2005) Am J Hum Genet 76; 887-893; Li M, Boehnke M, & Abecasis G R (2005) Am J Hum Genet 76; 934-949). To evaluate multi-SNP models, we first imputed missing genotypes were first imputed (See Scheet P & Stephens M (2006) Am J Hum Genet 78; 629-644).

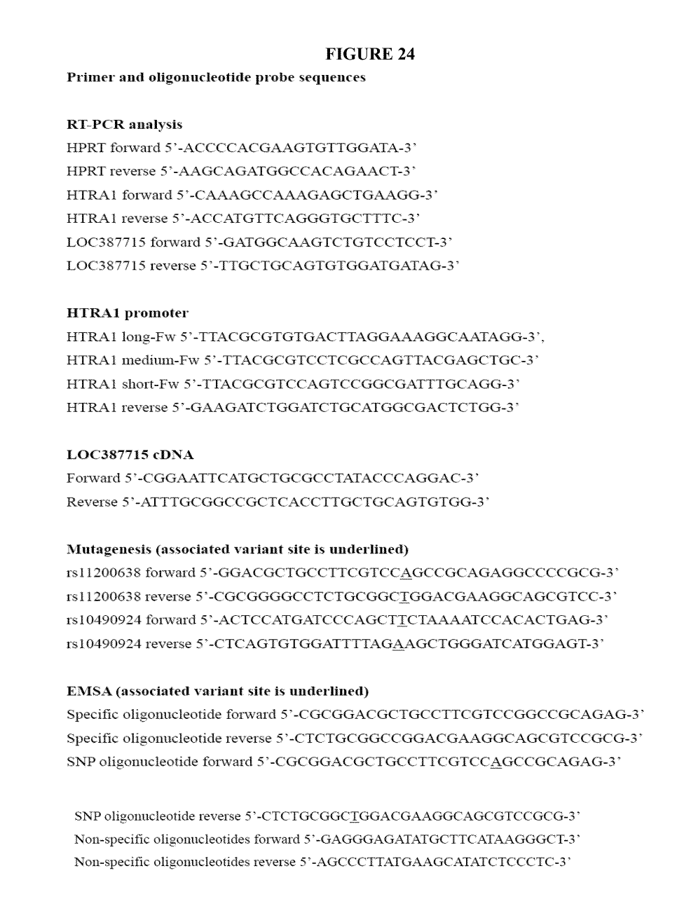

RT-PCR analysis. Human retina tissues were procured from National Disease Research Interchange, Philadelphia. Total RNA from retinas of 4 adults each with AMD (ages 60 to 93 yr) or without any maculopathy (ages 64 to 100 yr) was reverse transcribed per standard protocols (See Sambrook J & Russell D W (2001) Molercular Cloning, A Laboratory Mannual, Third Edition (Cold Spring Harbbor Laboratory Press, New York). qPCR reactions were performed in triplicate with Platinum Taq polymerase (Invitrogen) using the iCycler iQ Real-Time PCR Detection System (Biorad, Hercules, Calif.). SYBR Green I (Invitrogen) was used for detection, and results were analyzed by the ΔΔCt method using HPRT for normalization. Primers are listed in FIG. 24.

Plasmid construction and mutagenesis. Three regions of the HTRA1 promoter (−3652 to +57, −775 to +57, and −425 to +57) (GenBank accession #AF157623) were subcloned into pGL3-basic vector (Promega, Madison, Wis.). The full-length LOC387715/ARMS2 (XM_{—}001131263) cDNA was amplified from human retinal RNA by RT-PCR and cloned into pcDNA4 His/Max C vector (Invitrogen). The QuickChange XL site-directed mutagenesis kit (Stratagene, La Jolla, Calif.) was used to generate all mutants of the HTRA1 promoter and LOC387715/ARMS2 expression construct.

Electrophoretic mobility shift assays (EMSA). Nuclear extracts from bovine retina were used for EMSA per standard protocols (See Sambrook J & Russell D W (2001) Molercular Cloning, A Laboratory Mannual, Third Edition (Cold Spring Harbbor Laboratory Press, New York)). In super-shift experiments, antibodies against AP-2α and SP-1 (Santa Cruz Biotechnology Inc., Santa Cruz, Calif.), and NRL (a retina-pineal specific transcription factor) (See Swain P K, Hicks D, Mears A J, Apel I J, Smith J E, John S K, Hendrickson A, Milam A H, & Swaroop A (2001) J Biol Chem 276; 36824-36830) were added after the incubation of ³²P-labeled oligonucleotides with retinal nuclear extract.

Antibody generation. Rabbit anti-LOC387715/ARMS2 polyclonal antibody was raised against the linear peptide sequences ⁴⁷GGEGASDKQRSKL⁵⁹ and ⁸⁷QRRFQQPQHHLTLS¹⁰⁰, derived from the predicted human LOC387715/ARMS2 protein (XP_{—}001131263).

Transfections, protein analysis, and immunocytochemistry. Cells were cultured according to standard procedures and transfected at 80% confluency with plasmid DNA using FuGENE6 (Roche Applied Science, Indianapolis, Ind.). For luciferase assays, each plasmid containing pGL3-HTRA1 WT or SNP (0.5 μg per well) was co-transfected with cytomegalovirus-β-galactosidase (0.1 μg per well) plasmid to normalize for the amount of DNA and transfection efficiency, and the reporter activity was measured by a kit from Promega. Transfections were repeated in triplicate and three times. Cell extracts were subjected to immunoblotting using mouse monoclonal anti-Xpress antibody (Invitrogen), rabbit anti-cytochrome c oxidase IV (COX IV) (Abcam Inc., Cambridge, Mass.), or rabbit anti-Tom 20 antibody (Santa Cruz Biotechnology), according to the standard protocols (See Ausubel F M, Brent, R., Kingston, R. E., Moore, D. D., J. G., S., Smith, J. A., and Struhl, K. (1989) Current Protocols in Molecular Biology (New York).). Fractionation of COS-1 cell extracts was performed as described (See Bonifacino J S, Dasso, M., Harford, J. B., Lippincott-Schwartz, J., Yamada, K. M. (2007) Current Protocols in Cell Biology (John Wiley and Sons, Inc., New Jersey).). In some experiments, the mitochondrial fraction was treated with Proteinase K for 3 min at 26° C. Immunostaining was performed, as described (See Kanda A, Friedman J S, Nishiguchi K M, & Swaroop A (2007) Hum Mutat 28; 589-598), using anti-Xpress antibody, MitoTracker and LysoTracker (Molecular Probes, Eugene, Oreg.), rabbit anti-cytochrome c oxidase IV (COX IV) and rabbit anti-Giantin (Abcam Inc., Cambridge), and rabbit anti-protein disulfide isomerase antibody (PDI) (StressGen Biotechnologies, BC, Canada).

### Association Analysis

Genome-wide linkage studies have revealed disease susceptibility haplotypes of large effect at chromosomes 1q31-32 and 10q26 (See, e.g., Fisher S A, Abecasis G R, Yashar B M, Zareparsi S, Swaroop A, Iyengar S K, Klein B E, Klein R, Lee K E, Majewski J, et al. (2005) Hum Mol Genet 14; 2257-2264). In a remarkable example of the convergence of alternative approaches for gene mapping, independent research efforts identified the Y402H variant in complement factor H (CFH) on chromosome 1q32 as the first major AMD susceptibility allele (See, e.g., Klein R J, Zeiss C, Chew E Y, Tsai J Y, Sackler R S, Haynes C, Henning A K, SanGiovanni J P, Mane S M, Mayne S T, et al. (2005) Science 308; 385-389., Edwards A O, Ritter R, 3rd, Abel K J, Manning A, Panhuysen C, & Farrer L A (2005) Science 308; 421-424; Hageman G S, Anderson D H, Johnson L V, Hancox L S, Taiber A J, Hardisty L I, Hageman J L, Stockman H A, Borchardt J D, Gehrs K M, et al. (2005) Proc Natl Acad Sci USA 102; 7227-7232.; Haines J L, Hauser M A, Schmidt S, Scott W K, Olson L M, Gallins P, Spencer K L, Kwan S Y, Noureddine M, Gilbert J R, et al. (2005) Science 308; 419-421; Zareparsi S, Branham K E, Li M, Shah S, Klein R J, Ott J, Hoh J, Abecasis G R, & Swaroop A (2005) Am J Hum Genet 77; 149-153). A putative second genomic region with similarly consistent linkage evidence may exist at chromosome 10q26, where rs10490924 and nearby single-nucleotide polymorphisms (SNPs) that span a 200-kb region of linkage disequilibrium display association to AMD (See, e.g., Schmidt S, Hauser M A, Scott W K, Postel E A, Agarwal A, Gallins P, Wong F, Chen Y S, Spencer K, Schnetz-Boutaud N, et al. (2006) Am J Hum Genet 78; 852-864; Jakobsdottir J, Conley Y P, Weeks D E, Mah T S, Ferrell R E, & Gorin M B (2005) Am J Hum Genet 77; 389-407; Rivera A, Fisher S A, Fritsche L G, Keilhauer C N, Lichtner P, Meitinger T, & Weber B H (2005) Hum Mol Genet 14; 3227-3236). Markers showing evidence of association at 10q26 overlap with three genes, PLEKHA1, LOC387715/ARMS2 (Age-Related Maculopathy Susceptibility 2) and HTRA1/PRSS11 (High Temperature Requirement factor A1). PLEKHA1 has a pleckstrin homology domain, while LOC387715/ARMS2 encodes a hypothetical protein of unknown function. It was initially proposed that polymorphisms in the region alter the risk of AMD by modulating the function of one of these two genes (See, e.g., Schmidt S, Hauser M A, Scott W K, Postel E A, Agarwal A, Gallins P, Wong F, Chen Y S, Spencer K, Schnetz-Boutaud N, et al. (2006) Am J Hum Genet 78; 852-864; Jakobsdottir J, Conley Y P, Weeks D E, Mah T S, Ferrell R E, & Gorin M B (2005) Am J Hum Genet 77; 389-407; Rivera A, Fisher S A, Fritsche L G, Keilhauer C N, Lichtner P, Meitinger T, & Weber B H (2005) Hum Mol Genet 14; 3227-3236). More recently, two reports proposed a causal relationship between AMD susceptibility and rs11200638, another SNP in the same 200-kb region of 10q26, and suggested that this promoter variant affects the expression of a serine protease HTRA1/PRSS11 (See, e.g., Dewan A, Liu M, Hartman S, Zhang S S, Liu DT, Zhao C, Tam P O, Chan W M, Lam D S, Snyder M, et al. (2006) Science 314, 989-992). This interpretation contrasts sharply with other reports (See, e.g., Schmidt S, Hauser M A, Scott W K, Postel E A, Agarwal A, Gallins P, Wong F, Chen Y S, Spencer K, Schnetz-Boutaud N, et al. (2006) Am J Hum Genet 78; 852-864; Jakobsdottir J, Conley Y P, Weeks D E, Mah T S, Ferrell R E, & Gorin M B (2005) Am J Hum Genet 77; 389-407; Rivera A, Fisher S A, Fritsche L G, Keilhauer C N, Lichtner P, Meitinger T, & Weber B H (2005) Hum Mol Genet 14; 3227-3236), which find the strongest association with rs10490924; T allele of rs10490924 maps to exon 1 of the hypothetical LOC387715/ARMS2 gene and changes putative amino acid 69 from alanine to serine.

To resolve the sharply contradictory reports, a detailed association analysis of SNPs at 10q26 was undertaken. In some embodiments, the present invention provides strong association of AMD susceptibility to rs10490924 that cannot be explained by rs11200638. In some embodiments, the region surrounding the rs11200638 variant does not bind to AP-2α transcription factor and has no significant effect on HTRA1 mRNA expression. In some embodiments, the rs10490924 variant alters the coding sequence of a primate-specific gene LOC387715/ARMS2. In some embodiments, the present invention provides that LOC387715/ARMS2 produce a protein that localizes to the mitochondria when expressed in mammalian cells. In some embodiments, the present invention provides that changes in the activity and/or regulation of LOC387715/ARMS2 are responsible for the impact of rs10490924 on AMD disease susceptibility, and that the association of AMD with rs11200638 is indirect

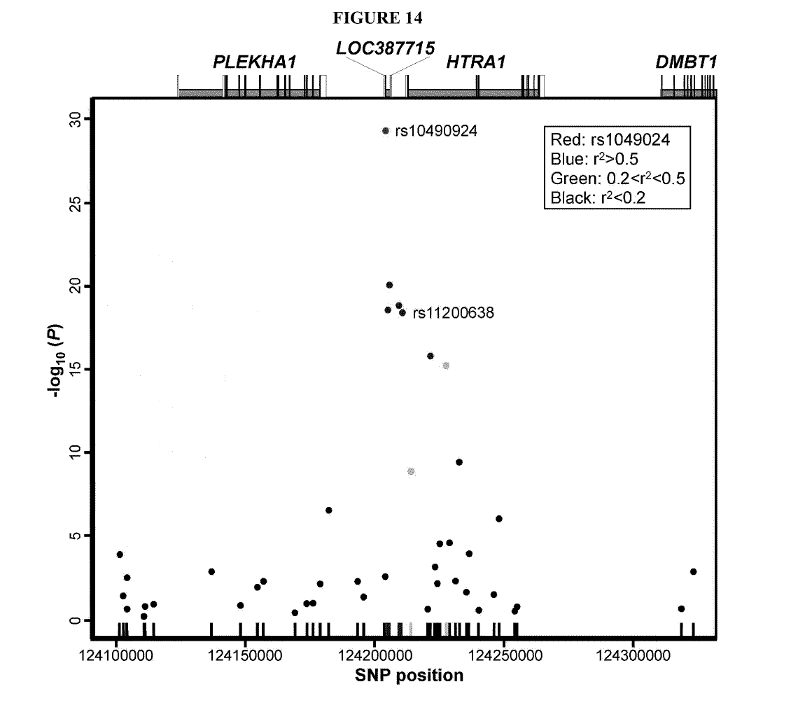

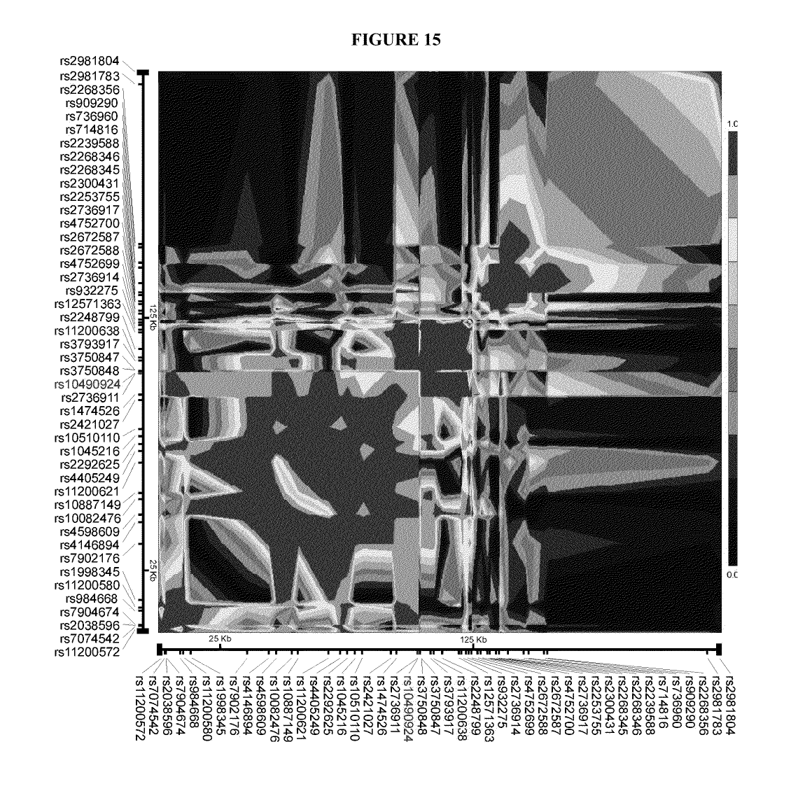

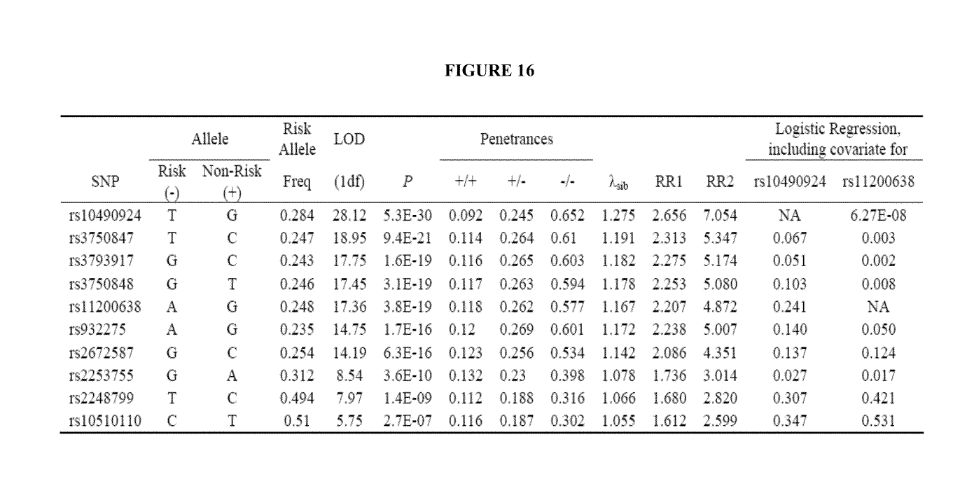

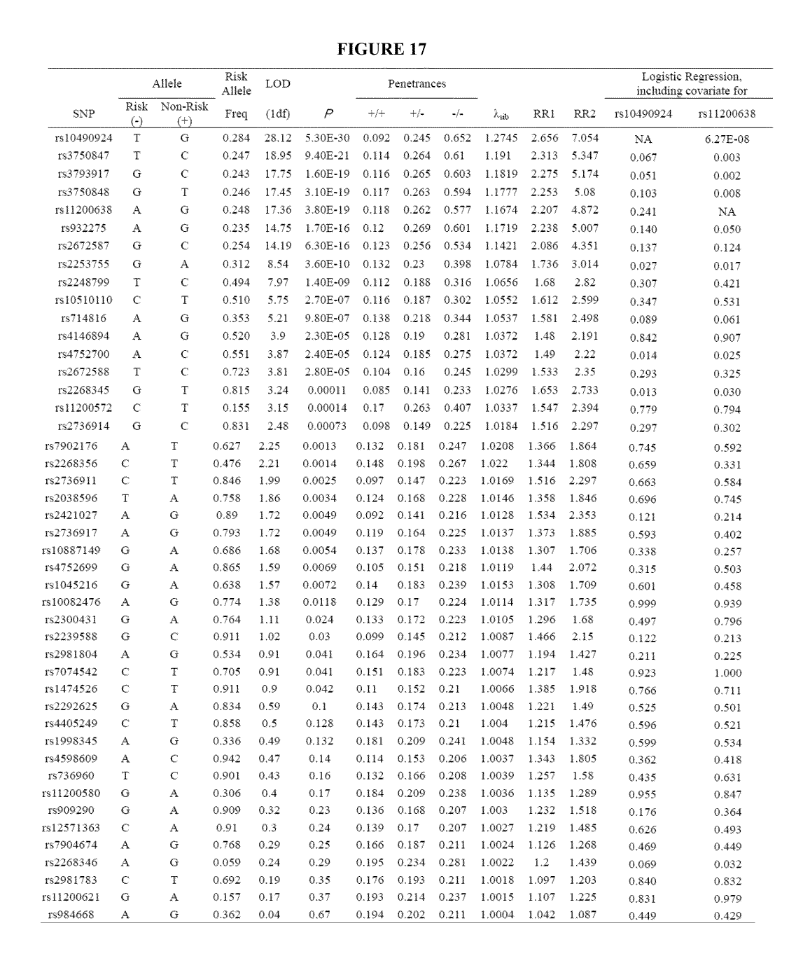

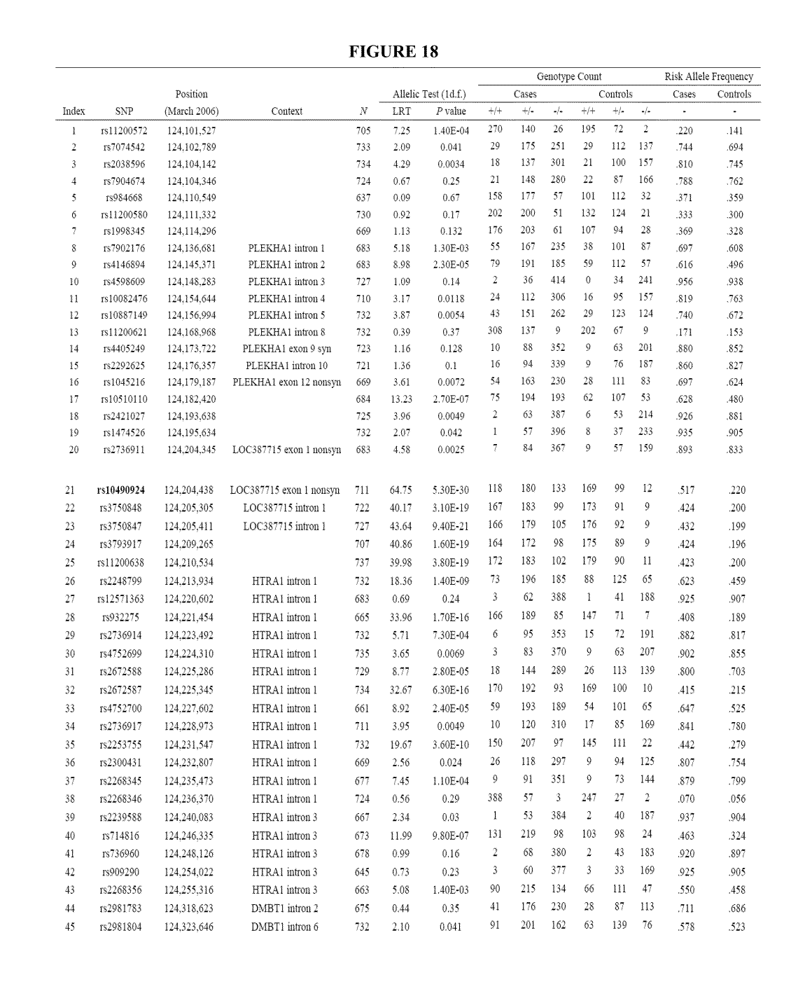

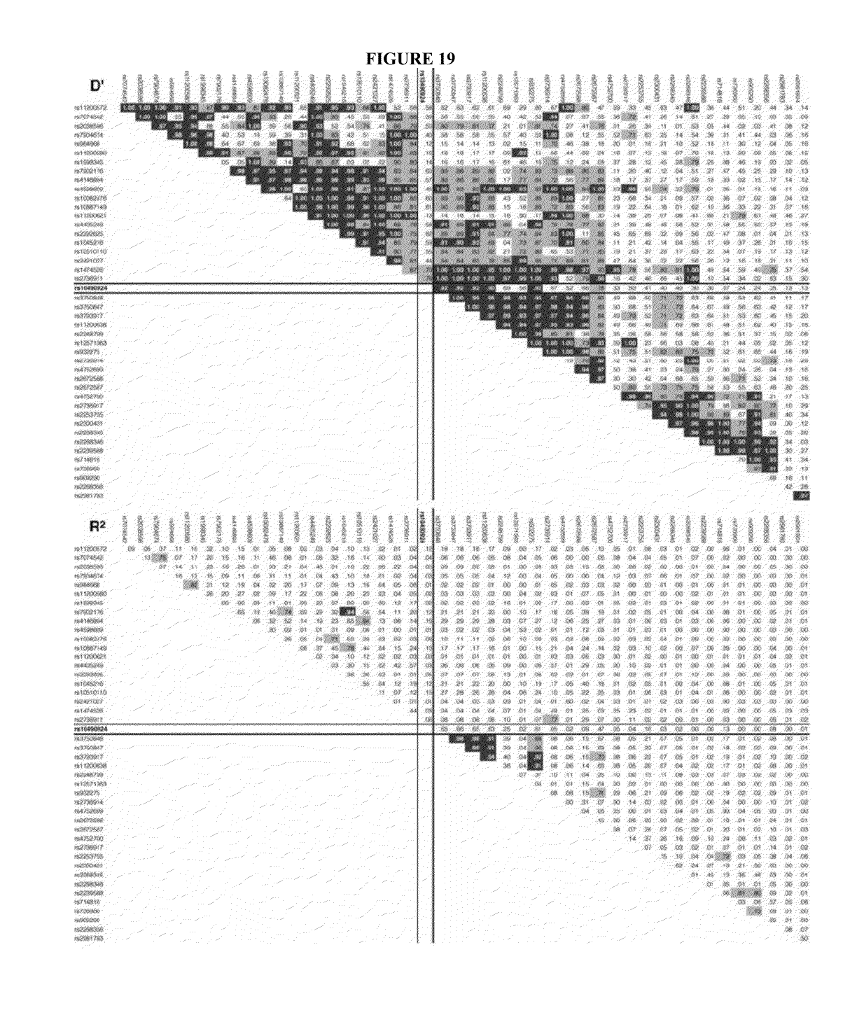

In order to examine the association of rs10490924, rs11200638, and neighboring variants with AMD, these two and an additional 43 SNPs in a cohort of 466 AMD cases and 280 controls were genotyped. The SNPs were selected to capture 172 common polymorphisms characterized by the HapMap consortium (See (2005) Nature 437; 1299-1320) in the 220-kb region spanning PLEKHA1, LOC387715/ARMS2 and HTRA1 with an average r² of 0.92. The results are summarized in FIGS. 14-15 the top 10 SNPs in FIG. 16, and FIGS. 17-18. After fitting a parametric association model (See, e.g., Li M, Atmaca-Sonmez P, Othman M, Branham K E, Khanna R, Wade M S, Li Y, Liang L, Zareparsi S, Swaroop A, et al. (2006) Nat Genet 38; 1049-1054.; Li M, Boehnke M, & Abecasis G R (2005) Am J Hum Genet 76; 934-949), marker rs10490924 showed the strongest association with AMD (P=5.3*10⁻³⁰), with an estimated relative risk of 2.66 for GT heterozygotes and 7.05 for TT homozygotes. The risk allele T has a significantly higher frequency in cases than in controls (51.7% vs 22.0%, P<10⁻²⁸). Four other SNPs (rs3750847, rs3793917, rs3750848, rs11200638) show strong but less significant association (10⁻²¹<P<10⁻¹⁸). In particular, the rs11200638 SNP showed a weaker association (P=3.8*10⁻¹⁹) with an estimated relative risk of 2.21 for AG heterozygotes and 4.87 for AA homozygotes. The five listed SNPs are in high linkage disequilibrium (See FIGS. 14 and 19). Using logistic regression to evaluate models with two or more SNPs, it was determined that when rs10490924 was included no other SNP showed significant evidence for association (rs2253755 had the strongest association after accounting for rs10490924, P=0.027, which is non-significant after adjusting for multiple testing). In contrast, when rs11200638 or any other SNP was used to seed the model, rs10490924 still showed significant evidence for association (P<10⁻⁶ or less, depending on the SNP used to seed the model). Overall, the genetic data is consistent with a model where rs10490924 alone, or another ungenotyped SNP in very strong disequilibrium with it, is directly responsible for association with AMD. In addition, the results provide that rs11200638 and the other examined SNPs are only indirectly associated with the disease. The data does not support a model where rs11200638 alone explains the association of the 10q26 region with macular degeneration.

In addition to a multiplicative model with one degree of freedom (as outlined above), models with two degree of freedom were also fitted to the data. These models did not significantly improve fit (P>0.1) and did not lead to qualitatively different conclusions. In particular, the data still led to the conclusion that rs10490924 was the strongest associated SNP in the region and that association with any other SNP could be accounted for by rs10490924. These two degree of freedom also did not support the possibility that rs11200638 is the major determinant of disease susceptibility in the region.

Effect of rs11200638 on HTRA1 Expression.

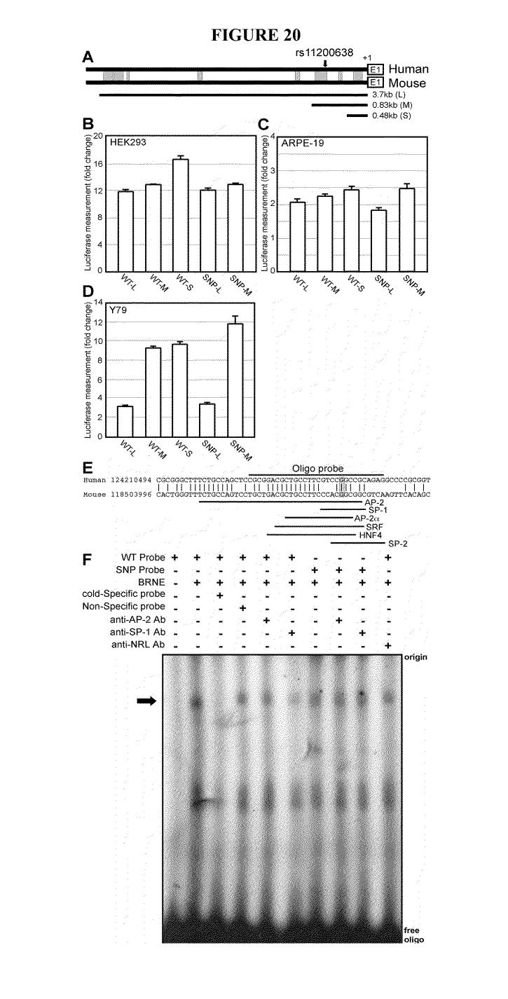

The impact of the previously-proposed causal variant rs11200638 on HTRA1 expression were examined and the potential roles of LOC387715/ARMS2 (the hypothetical gene whose coding sequence is altered by rs10490924) investigated. The SNP rs11200638 is located within a conserved genomic region upstream of human and mouse HTRA1 genes (See FIG. 20A). To evaluate previous reports (See, e.g., Dewan A, Liu M, Hartman S, Zhang S S, Liu D T, Zhao C, Tam P O, Chan W M, Lam D S, Snyder M, et al. (2006) Science 314; Yang Z, Camp N J, Sun H, Tong Z, Gibbs D, Cameron D J, Chen H, Zhao Y, Pearson E, Li X, et al. (2006) Science 314; 992-993) of the effects of SNP rs11200638 on HTRA1 promoter activity, mammalian expression constructs were generated carrying three different lengths of the wild-type HTRA1 promoter (WT-long, -medium, and -short) and the mutant sequence carrying the AMD risk allele at the SNP rs11200638 (SNP-long and -medium). These constructs were transfected into HEK293 (human embryonic kidney), ARPE-19 (human RPE), and Y79 (human retinoblastoma) cells; in all three cell lines, WT and variant SNP promoter activities did not show statistically significant differences in the luciferase reporter expression, and the WT-short promoter (not including rs11200638 region) showed higher transcriptional activities than the others (See FIG. 20B-D).

Although the rs11200638 region includes several transcription factor binding sites as suggested by in silico analysis (See FIG. 20E), Dewan et al. focused on putative binding sites for transcription factors activating enhancer-binding protein-2α (AP-2α) and serum response factor (See Dewan A, Liu M, Hartman S, Zhang S S, Liu D T, Zhao C, Tam P O, Chan W M, Lam D S, Snyder M, et al. (2006) Science 314). Electrophoretic mobility shift assays (EMSA) did not detect any supershift of the nucleotide sequence spanning rs11200638 variation with anti-AP-2α antibody (See FIG. 20F, lane 5). Among the transcription factors examined, only stimulating protein 1 (SP-1) antibody produced a weakly-shifted DNA-protein complex (See FIG. 20F, lane 6). Quantitative RT-PCR analysis provided suggestive evidence for a decrease in HTRA1 expression in AMD retinas (similar threshold levels after an average of 21.6±0.6 RT-PCR cycles in control retinas versus 22.2±0.3 cycles in AMD retinas; 4 independent retinas examined in quadruplicate for each). This contrasts with the smaller original experiment suggesting an increase in HTRA1 expression in lymphocytes from AMD patients (p=0.02) (See, e.g., Dewan A, Liu M, Hartman S, Zhang S S, Liu D T, Zhao C, Tam P O, Chan W M, Lam D S, Snyder M, et al. (2006) Science 314; Yang Z, Camp N J, Sun H, Tong Z, Gibbs D, Cameron D J, Chen H, Zhao Y, Pearson E, Li X, et al. (2006) Science 314; 992-993). Taken together, the present invention provides that there is no significant change in HTRA1 expression between AMD patients and controls.

### Expression and Subcellular Localization of LOC387715/ARMS2

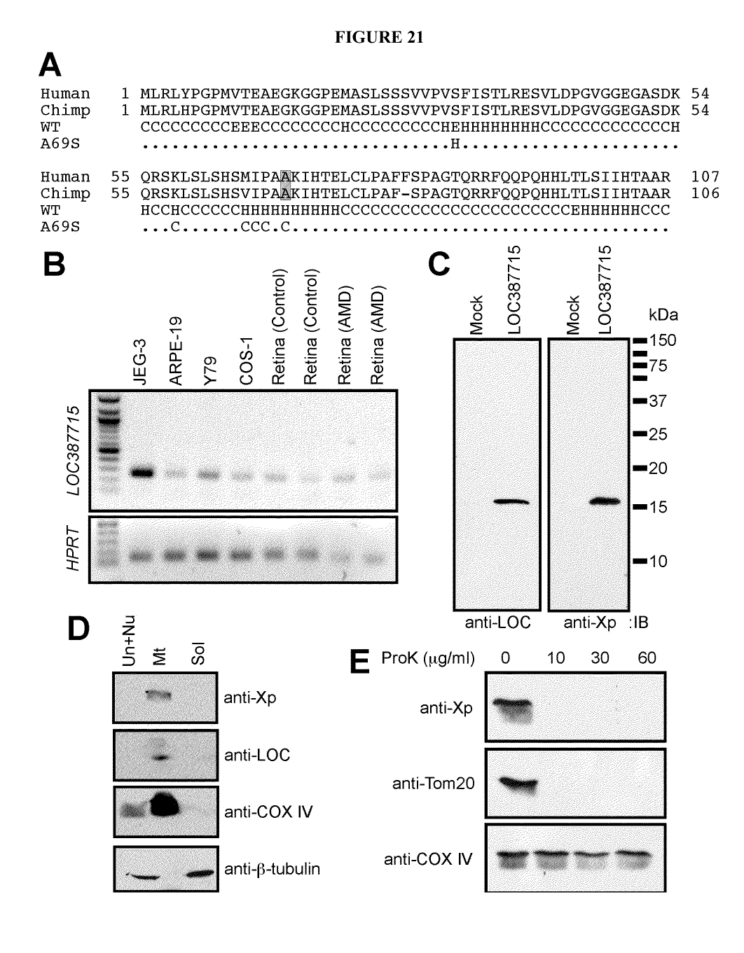

The possible role of LOC387715/ARMS2, the hypothetical gene whose coding sequence is altered by rs10490924, was investigated. LOC387715/ARMS2 encodes a predicted human protein with a highly-conserved ortholog in chimpanzee, but not in other mammals or vertebrates (See FIG. 21A). The T allele of SNP rs10490924 is predicted to result in a coding change (A69S) of the LOC387715/ARMS2 protein. This alanine to serine substitution creates a new putative phosphorylation site and breaks a predicted α-helix (See FIG. 21A).

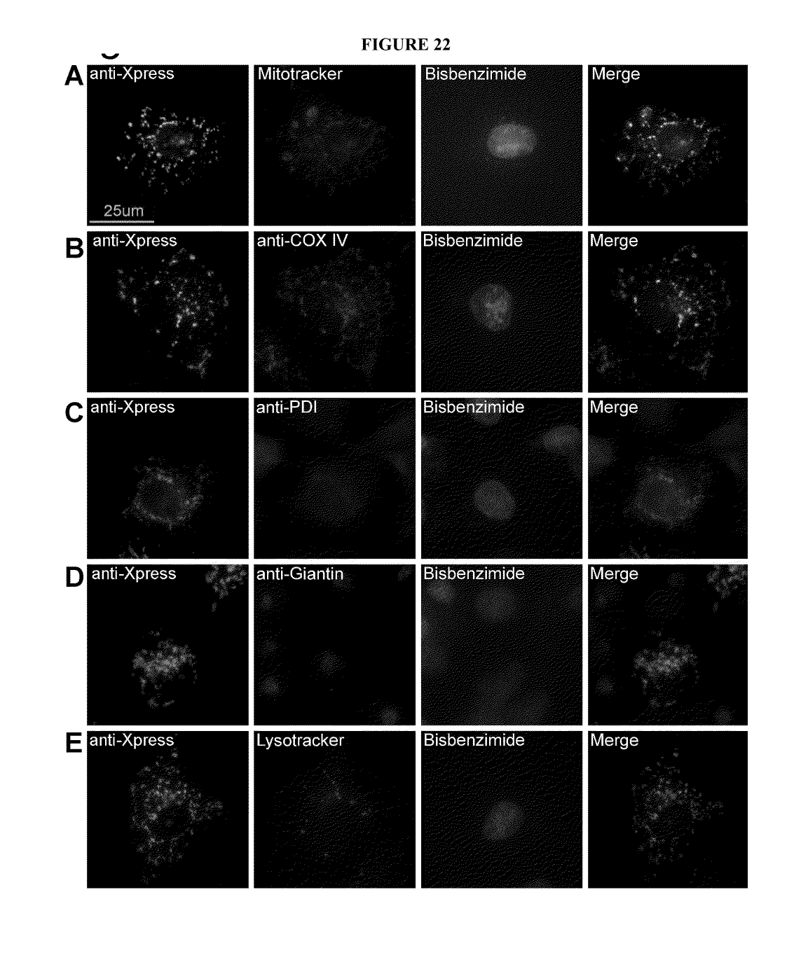

RT-PCR analysis showed that LOC387715/ARMS2 mRNA is expressed abundantly in JEG-3 (human placenta choriocarcinoma) and faintly in the human retina and other cell lines, whereas HPRT (control) transcript is detected to a similar degree in all tissues/cell lines (See FIG. 21B). Using the human retinal RNA, the LOC387715/ARMS2 cDNA was cloned into an expression vector and expressed it in COS-1 (African green monkey kidney fibroblast) cells. Immunoblot analysis revealed a predicted protein band of approximately 16 kDa (12 kDa protein+4 kDa Xpress epitope) using anti-Xpress and anti-LOC387715/ARMS2 antibodies (See FIG. 21C). Subcellular fractionation and co-staining patterns of MitoTracker and cytochrome c oxidase subunit IV (COX IV) demonstrated that the expressed LOC387715/ARMS2 protein co-localizes with mitochondrial markers, but not with other organelle markers for endoplasmic reticulum (ER), Golgi apparatus, and lysosomes (See FIG. 21D, and FIG. 22A-E). Similar results were obtained in the ARPE-19 and JEG-3 cells. The treatment of mitochondrial protein fraction, prepared from the transfected COS-1 cells, with Proteinase K resulted in the loss of LOC387715/ARMS2 as well as outer membrane proteins (such as translocase of outer mitochondrial membrane 20, Tom20), with no effect on COX-IV, an inner membrane protein (See FIG. 21E).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### REFERENCES

1. Majewski, J. et al. Age-related macular degeneration—a genome scan in extended families. Am. J. Hum. Genet. 73, 540-550 (2003).
2. Abecasis, G. R. et al. Age-related macular degeneration: a high-resolution genome scan for susceptibility loci in a population enriched for late-stage disease. Am. J. Hum. Genet. 74, 482-494 (2004).
3. Weeks, D. E. et al. Age-related maculopathy: an expanded genome-wide scan with evidence of susceptibility loci within the 1q31 and 17q25 regions. Am. J. Ophthalmol. 132, 682-692 (2001).
4. Seddon, J. M., Santangelo, S. L., Book, K., Chong, S. & Cote, J. A genomewide scan for age-related macular degeneration provides evidence for linkage to several chromosomal regions. Am. J. Hum. Genet. 73, 780-790 (2003).
5. Fisher, S. A. et al. Meta-analysis of genome scans of age-related macular degeneration. Hum. Mol. Genet. 14, 2257-2264 (2005).
6. Hirvela, H., Luukinen, H., Laara, E., Sc, L. & Laatikainen, L. Risk factors of age-related maculopathy in a population 70 years of age or older. Ophthalmology 103, 871-877 (1996).
7. Smith, W. et al. Risk factors for age-related macular degeneration: Pooled findings from three continents. Ophthalmology 108, 697-704 (2001).
8. Klein, R., Klein, B. E., Tomany, S. C. & Moss, S. E. Ten-year incidence of age-related maculopathy and smoking and drinking: the Beaver Dam Eye Study. Am. J. Epidemiol. 156, 589-598 (2002).
9. Schmidt, S. et al. Cigarette smoking strongly modifies the association of LOC387715 and age-related macular degeneration. Am. J. Hum. Genet. 78, 852-864 (2006).
10. Klein, R. J. et al. Complement factor H polymorphism in age-related macular degeneration. Science 308, 385-389 (2005).
11. Haines, J. L. et al. Complement factor H variant increases the risk of age-related macular degeneration. Science 308, 419-421 (2005).
12. Edwards, A. O. et al. Complement factor H polymorphism and age-related macular degeneration. Science 308, 421-424 (2005).
13. Jakobsdottir, J. et al. Susceptibility genes for age-related maculopathy on chromosome 10q26. Am. J. Hum. Genet. 77, 389-407 (2005).
14. Rivera, A. et al. Hypothetical LOC387715 is a second major susceptibility gene for age-related macular degeneration, contributing independently of complement factor H to disease risk. Hum. Mol. Genet. 14, 3227-3236 (2005).
15. Zareparsi, S. et al. Strong association of the Y402H variant in complement factor H at 1q32 with susceptibility to age-related macular degeneration. Am. J. Hum. Genet. 77, 149-153 (2005).
16. Li, M., Boehnke, M. & Abecasis, G. R. Joint modeling of linkage and association: identifying SNPs responsible for a linkage signal. Am. J. Hum. Genet. 76, 934-949 (2005).
17. Li, M., Boehnke, M. & Abecasis, G. R. Efficient study designs for test of genetic association using sibship data and unrelated cases and controls. Am. J. Hum. Genet. 78, 778-792 (2006).
18. Risch, N. Linkage strategies for genetically complex traits. I. Multilocus models. Am. J. Hum. Genet. 46, 222-228 (1990).
19. Hodge, S. E. & Elston, R. C. Lods, wrods, and mods: the interpretation of lod scores calculated under different models. Genet. Epidemiol. 11, 329-342 (1994).
20. Valdes, A. M. & Thomson, G. Detecting disease-predisposing variants: the haplotype method. Am. J. Hum. Genet. 60, 703-716 (1997).
21. Zaykin, D. V. et al. Testing association of statistically inferred haplotypes with discrete and continuous traits in samples of unrelated individuals. Hum. Hexed. 53, 79-91 (2002).
22. Stephens, M., Smith, N. J. & Donnelly, P. A new statistical method for haplotype reconstruction from population data. Am. J. Hum. Genet. 68, 978-989 (2001).
23. Li, N. & Stephens, M. Modeling linkage disequilibrium and identifying recombination hotspots using single-nucleotide polymorphism data. Genetics 165, 2213-2233 (2003).
24. The International HapMap Consortium. The International HapMap Project. Nature 437, 1299-1320 (2005).
25. Monks, S. A. et al. Genetic inheritance of gene expression in human cell lines. Am. J. Hum. Genet. 75, 1094-1105 (2004).
26. Bird, A. C. et al. An international classification and grading system for age-related maculopathy and age-related macular degeneration. The International ARM Epidemiological Study Group. Surv. Ophthalmol. 39, 367-374 (1995).
27. Wigginton, J. E., Cutler, D. J. & Abecasis, G. R. A note on exact tests of Hardy-Weinberg equilibrium. Am. J. Hum. Genet. 76, 887-883 (2005).
28. Abecasis, G. R., Martin, R. & Lewitzky, S. Estimation of haplotype frequencies from diploid data. Am. J. Hum. Genet. 69, 5198 (2001).
29. Abecasis, G. R. & Cookson, W.O.C. GOLD—graphical overview of linkage disequilibrium. Bioinformatics 16, 182-183 (2000).
30. Stephens, M. & Scheet, P. Accounting for decay of linkage disequilibrium in haplotype inference and missing-data imputation. Am. J. Hum. Genet. 76, 449-462 (2005).

1.-16. (canceled)

17. A method for identifying a human subject's risk for developing age-related macular degeneration (AMD) comprising:
(a) detecting in vitro the presence of an A allele of rs1280514 from a sample from said human subject,
(b) detecting in vitro the presence of a C allele of rs3766405 from said sample from said human subject, and
(c) diagnosing said human subject as having an increased risk of AMD based on the presence of said A allele of rs1280514 and the presence of said C allele of rs3766405.

18. The method of claim 17, further comprising detecting the presence of a C allele of rs412852.

19. The method of claim 17, further comprising detecting the presence of a C allele of rs11582939.

20. The method of claim 17, further comprising detecting the presence of a G allele of rs1048663.

21. The method of claim 17, further comprising detecting the presence of a C allele of rs412852, a C allele of rs11582939, and a G allele of rs1048663.

22. The method of claim 17, further comprising detecting polymorphisms and/or variants found in LOC387715/ARMS2.

23. The method of claim 21, further comprising detecting polymorphisms and/or variants found in LOC387715/ARMS2.

24. The method of claim 17, wherein said subject is a subject suspected of having AMD.

25. The method of claim 17, wherein said subject is a subject diagnosed with AMD.

26. The method of claim 17, wherein said subject is at risk for AMD.

27. The method of claim 17, wherein said detecting comprises amplification of nucleic acid.

28. The method of claim 17, wherein said detecting comprises nucleic acid sequencing.

29. The method of claim 17, wherein said sample is a biological fluid sample.

30. The method of claim 17, wherein said sample comprises a blood product.

31. The method of claim 17, further comprising the step of selecting and administering a treatment to said subject based on said detecting.

32. The method of claim 17, further comprising detecting the presence of a C allele of rs2274700.

33. The method of claim 17, further comprising detecting the presence of an A allele of rs1061147.

34. The method of claim 17, further comprising detecting the presence of a C allele of rs1061170.

35. A method for determining a human subject's genetic predisposition for developing age-related macular degeneration (AMD) in a human subject, said method comprising:
(a) detecting in vitro the presence of an A allele or G allele of rs1280514 in a sample from a human subject
(b) correlating the presence of said A allele of rs1280514 with the presence of an increased genetic predisposition related to rs1280514 for developing AMD in said human subject, or the presence of said G allele of rs1280514 with the absence of an increased genetic predisposition related to rs1280514 for developing AMD in said human subject;
(c) detecting in vitro the presence of a C allele of rs3766405 from said sample from said human subject;
(d) correlating the presence of a C allele of rs3766405 with the presence of an increased genetic predisposition related to rs3766405 for developing AMD-in said human subject;
(e) determining said human subject's risk of developing AMD based on steps (a)-(d).

36. The method of claim 35, further comprising one or more of:
(i) detecting the presence of a C allele of rs412852, and correlating the presence of a C allele of rs412852 with the presence of an increased genetic predisposition for developing AMD in said human subject;
(ii) detecting the presence of a C allele of rs11582939, and correlating the presence of a C allele of rs11582939 with the presence of an increased genetic predisposition for developing AMD-in said human subject;
(iii) detecting the presence of a G allele of rs1048663, and correlating the presence of a G allele of rs1048663 with the presence of an increased genetic predisposition for developing AMD-in said human subject; and
(iv) detecting the presence of a C allele of rs3766405, and correlating the presence of a C allele of rs3766405 with the presence of an increased genetic predisposition for developing AMD in said human subject.


SEQUENCE LISTING (39 sequences)

SEQ ID NO: 1 - protein, 13 aa, Artificial Sequence
  Synthetic
GGEGASDKQRSKL

SEQ ID NO: 2 - protein, 14 aa, Artificial Sequence
  Synthetic
QRRFQQPQHHLTLS

SEQ ID NO: 3 - DNA, 60 bp, Artificial Sequence
  Synthetic
cgcgggctttctgccagctccgcggacgctgccttcgtccggccgcagaggccccgcggt

SEQ ID NO: 4 - DNA, 60 bp, Artificial Sequence
  Synthetic
cactgggtttctgccagtcctgctgacgctgccttcccacggcggcgtcaagttcacagc

SEQ ID NO: 5 - protein, 107 aa, Artificial Sequence
  Synthetic
MLRLYPGPMVTEAEGKGGPEMASLSSSVVPVSFISTLRESVLDPGVGGEGASDKQRSKLSLSHSMIPAAKIHTELCLPAFFSPAGTQRRFQQPQHHLTLSIIHTAAR

SEQ ID NO: 6 - protein, 106 aa, Artificial Sequence
  Synthetic
MLRLHPGPMVTEAEGKGGPEMASLSSSVVPVSFISTLRESVLDPGVGGEGASDKQRSKLSLSHSVIPAAKIHTELCLPAFSPAGTQRRFQQPQHHLTLSIIHTAAR

SEQ ID NO: 7 - protein, 107 aa, Artificial Sequence
  Synthetic
CCCCCCCCCEEECCCCCCCCHCCCCCCCCCHEHHHHHHHHCCCCCCCCCCCCCHHCCHCCCCCCHHHHHHHHHHCCCCCCCCCCCCCCCCCCCCCCCEHHHHHHCCC

SEQ ID NO: 8 - DNA, 39 bp, Artificial Sequence
  Synthetic
tgtaaaacgacggccagtcggatgcaccaaagattctcc

SEQ ID NO: 9 - DNA, 41 bp, Artificial Sequence
  Synthetic
aggaaacagctatgaccatttcgcgtccttcaaactaatgg

SEQ ID NO: 10 - DNA, 20 bp, Artificial Sequence
  Synthetic
taccatcaggttcgactgga

SEQ ID NO: 11 - DNA, 22 bp, Artificial Sequence
  Synthetic
ggaagccacttcttcccctgac

SEQ ID NO: 12 - DNA, 24 bp, Artificial Sequence
  Synthetic
cccacttaatgtctatagggtgtg

SEQ ID NO: 13 - DNA, 21 bp, Artificial Sequence
  Synthetic
gctacagaaaagccctcaggt

SEQ ID NO: 14 - DNA, 20 bp, Artificial Sequence
  Synthetic
tgcaaacgttccctattggt

SEQ ID NO: 15 - DNA, 20 bp, Artificial Sequence
  Synthetic
atgaacatgccagggaaaag

SEQ ID NO: 16 - DNA, 19 bp, Artificial Sequence
  Synthetic
tgagtgagatggcagctgg

SEQ ID NO: 17 - DNA, 20 bp, Artificial Sequence
  Synthetic
tccagctattcaaccagagg

SEQ ID NO: 18 - DNA, 20 bp, Artificial Sequence
  Synthetic
accccacgaagtgttggata

SEQ ID NO: 19 - DNA, 20 bp, Artificial Sequence
  Synthetic
aagcagatggccacagaact

SEQ ID NO: 20 - DNA, 20 bp, Artificial Sequence
  Synthetic
caaagccaaagagctgaagg

SEQ ID NO: 21 - DNA, 20 bp, Artificial Sequence
  Synthetic
accatgttcagggtgctttc

SEQ ID NO: 22 - DNA, 20 bp, Artificial Sequence
  Synthetic
gatggcaagtctgtcctcct

SEQ ID NO: 23 - DNA, 21 bp, Artificial Sequence
  Synthetic
ttgctgcagtgtggatgatag

SEQ ID NO: 24 - DNA, 30 bp, Artificial Sequence
  Synthetic
ttacgcgtgtgacttaggaaaggcaatagg

SEQ ID NO: 25 - DNA, 28 bp, Artificial Sequence
  Synthetic
ttacgcgtcctcgccagttacgagctgc

SEQ ID NO: 26 - DNA, 28 bp, Artificial Sequence
  Synthetic
ttacgcgtccagtccggcgatttgcagg

SEQ ID NO: 27 - DNA, 29 bp, Artificial Sequence
  Synthetic
gaagatctggatctgcatggcgactctgg

SEQ ID NO: 28 - DNA, 30 bp, Artificial Sequence
  Synthetic
cggaattcatgctgcgcctatacccaggac

SEQ ID NO: 29 - DNA, 31 bp, Artificial Sequence
  Synthetic
atttgcggccgctcaccttgctgcagtgtgg

SEQ ID NO: 30 - DNA, 35 bp, Artificial Sequence
  Synthetic
ggacgctgccttcgtccagccgcagaggccccgcg

SEQ ID NO: 31 - DNA, 35 bp, Artificial Sequence
  Synthetic
cgcggggcctctgcggctggacgaaggcagcgtcc

SEQ ID NO: 32 - DNA, 35 bp, Artificial Sequence
  Synthetic
actccatgatcccagcttctaaaatccacactgag

SEQ ID NO: 33 - DNA, 35 bp, Artificial Sequence
  Synthetic
ctcagtgtggattttagaagctgggatcatggagt

SEQ ID NO: 34 - DNA, 30 bp, Artificial Sequence
  Synthetic
cgcggacgctgccttcgtccggccgcagag

SEQ ID NO: 35 - DNA, 30 bp, Artificial Sequence
  Synthetic
ctctgcggccggacgaaggcagcgtccgcg

SEQ ID NO: 36 - DNA, 30 bp, Artificial Sequence
  Synthetic
cgcggacgctgccttcgtccagccgcagag

SEQ ID NO: 37 - DNA, 30 bp, Artificial Sequence
  Synthetic
ctctgcggctggacgaaggcagcgtccgcg

SEQ ID NO: 38 - DNA, 25 bp, Artificial Sequence
  Synthetic
gagggagatatgcttcataagggct

SEQ ID NO: 39 - DNA, 25 bp, Artificial Sequence
  Synthetic
agcccttatgaagcatatctccctc